(19) United States
(12) Patent Application Publication
(10) Pub. No.: US 2026/0263395 A1
(43) Pub. Date: Sep. 10, 2026

(54) MEDICAL PATCH COMPRISING CAPSAICIN

(71) Applicant: LTS LOHMANN THERAPIE-SYSTEME AG, Andernach (DE)

(72) Inventor: Eva-Marie PRINZ, Weißenthurm (DE)

(21) Appl. No.: 19/164,707

(22) PCT Filed: Mar. 15, 2024
(86) PCT No.: PCT/EP2024/056984

§ 371 Date: Sep. 12, 2025

(30) Foreign Application Priority Data
Mar. 15, 2023 (EP) 23162161.6

(51) Int. Cl. A61K 31/165; A61K 9/70

(52) U.S. Cl. CPC A61K 31/165; A61K 9/7069

(57) ABSTRACT

The present invention relates to a medical patch for the administration of capsaicin comprising an active agent-containing layer structure comprising capsaicin, such as a medical patch for use in a method of treatment as well as processes of manufacture of such a medical patch.

### TECHNICAL FIELD OF THE INVENTION

The present invention relates to a medical patch for the administration of capsaicin, wherein the medical patch comprises an active agent-containing layer comprising a silicone based polymer and a skin contact layer. Further, the present invention relates to methods of treatment and uses of the medical patch.

### BACKGROUND OF THE INVENTION

Capsaicin ((6E)-N-[(4-hydroxy-3-methoxyphenyl)methyl]-8-methylnon-6-enamide), the major bioactive component of chili peppers, is an alkaloid found in the Capsicum family. It is a potent agonist of the transient receptor potential cation channel subfamily V member 1 (TRPV1), better known as vanilloid receptor. By binding to the TRPV1 receptor, the capsaicin molecule produces similar sensations to those of excessive heat or abrasive damage.

The burning and painful sensations associated with capsaicin result from its chemical interaction with sensory neurons expressing TRPV1. This receptor is a nonselective cation channel that allows the transient influx of Ca²⁺ when activated during the detection and transduction of nociceptive stimuli. As calcium in the cell is one of the most versatile second messengers in numerous intracellular signaling pathways and TRPV1 permits cations to pass through the cell membrane and into the cell when activated, the resulting depolarization of the neurons stimulates it to signal the brain. Thus, TRPV1 plays a critical role in pain signaling.

Capsaicin has a role as a non-narcotic analgesic and is currently used for the treatment of several pain syndromes such as neuropathic pain. Such pain is believed to result from sensitization reactions in the peripheral and central nervous system and can occur as a result of peripheral injuries, or as a result of systemic diseases such as HIV, herpes zoster syphilis, autoimmune diseases and diabetes. Furthermore, capsaicin has also demonstrated beneficial effect on osteoarthritis pain relief due to its high capacity to inhibit P substance release, a powerful neuropeptide pain neuromodulator from the sensory nerves to the central nervous system. In addition, capsaicin is suggested to be able to kill cancer cells by causing them to undergo apoptosis.

Capsaicin is most often used as a topical analgesic and exists in many formulations of cream, liquid and patch preparations of various strengths. For example, an 8% capsaicin patch dermal delivery system is administered under the brand name QUTENZA® (Grünenthal). The topical system is indicated in adults for the treatment of neuropathic pain associated with postherpetic neuralgia (PHN) as well as for neuropathic pain associated with diabetic peripheral neuropathy (DPN) of the feet, and is currently approved for use in adults in the treatment of post-surgical neuropathic pain (PSNP). QUTENZA® delivers prescription-strength capsaicin directly to the skin for reversibly desensitizing and defunctionalizing the TRPV1 receptor, and can thus provide sustained pain relief that lasts for up to three months.

However, capsaicin is a strong irritant and the most common adverse reactions occurring during QUTENZA® application include applications site reactions, such as erythema, pain and pruritus. In addition, it is known in the art that dermal delivery systems may cause skin irritations depending on the adhesive layer.

It is therefore desirable to provide a medical patch for the topical and/or transdermal delivery of capsaicin, which overcomes the problem of skin irritation, but nevertheless has advantageous properties regarding the release of the active agent as well as the adhesiveness.

### OBJECTS AND SUMMARY OF THE INVENTION

It is an object of the present invention to provide a medical patch for the administration of capsaicin, which is improved in comparison to the patches described in the prior art.

It is a further object of the present invention to provide a medical patch for the administration of capsaicin, which reduces the problem of skin irritation.

It is a further object of the present invention to provide a medical patch for the administration of capsaicin, providing a permeation rate which is sufficient for achieving a therapeutically effective dose. In particular, the object is to provide a medical patch for the administration of capsaicin, enabling a faster onset of action associated with a reduced lag time.

It is also an object of the present invention to provide a medical patch for the administration of capsaicin, which has improved adhesive properties. In particular, the object is to provide a medical patch for the administration of capsaicin, which complies with the needs of a convenient application in view of the wearing comfort as well as a clean and painless removal, and with regard to multiple applications in case of repositioning.

It is another object of the present invention to provide a medical patch for the administration of capsaicin, which can be used in a method of treatment.

These objects and others are accomplished by the present invention, which according to one aspect relates to a medical patch for the administration of capsaicin comprising an active agent-containing layer structure, said active agent-containing layer structure comprising:
A) a backing layer;
B) an active agent-containing layer comprising
   (i) capsaicin, and
   (ii) at least one silicone-based polymer;
and
C) a skin contact layer;
   wherein the skin contact layer is an adhesive layer comprising a polymer II (the at least one silicone-based polymer in the active agent-containing layer is also referred to as polymer I). In another aspect, the present invention relates to a medical patch for the administration of capsaicin comprising an active agent-containing layer structure, said active agent-containing layer structure comprising:
A) a backing layer;
B) an active agent-containing layer comprising
   (i) capsaicin, and
   (ii) at least one silicone-based polymer;
and
C) a skin contact layer;
   wherein the skin contact layer is an adhesive layer comprising a silicone gel adhesive.

It has been surprisingly found that the medical patch according to the present invention, which comprises a skin contact layer, in which the capsaicin is sparingly soluble and which is preferably directly attached to an active agent-containing layer, has advantageous properties regarding reduced skin irritation, but at the same time has improved drug delivery behavior and adhesive properties. In particular, since the capsaicin is sparingly soluble, e.g., has a saturation concentration of less than 0.1% in the skin contact layer, only an insignificant amount of the capsaicin is present on the surface of the skin contact layer so that the medical patch has advantageous properties regarding unwanted skin reactions and thus enables safe application and/or removal. Thus, such an inventive medical patch with a skin contact layer, in which the saturation concentration of the capsaicin is negligible, with a separated active agent-containing layer (not being in skin contact), prohibits the capsaicin to be released before and/or after the medical patch is applied to and maintained on the skin of the patient. On the other hand, it has been found that such a medical patch with a skin contact layer is still able to provide a sufficient drug delivery and might even allow for a faster release of active. Such inventive capsaicin medical patches are also advantageous in that they could be based on already approved and marketed capsaicin patches, which are merely supplemented with a further skin contact layer. Since the drug delivery is sufficiently similar, the prospect for being able to achieve bioequivalence to the commercial product is excellent, which is highly desirable from a regulatory point of view.

According to one specific aspect, the invention relates to a medical patch for the administration of capsaicin comprising an active agent-containing layer structure, said active agent-containing layer structure comprising:
A) a backing layer;
B) an active agent-containing layer comprising
   (i) capsaicin in an amount of from 5 to 10% by weight,
   (ii) at least one amine-compatible polysiloxane in an amount of from 60 to 90% by weight,
   (iii) diethylene glycol monoethyl ether in an amount of from 10 to 25% by weight,
   (iv) ethyl cellulose in an amount of from 0 to 2% by weight, and
   (v) silicone oil in amount of from 0 to 5% by weight;
and
C) a skin contact layer;
   wherein the skin contact layer is an adhesive layer comprising a silicone gel adhesive, and wherein the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.

According to another specific aspect, the invention relates to a medical patch for the administration of capsaicin comprising an active agent-containing layer structure, said active agent-containing layer structure comprising:
A) a backing layer;
B) an active agent-containing layer comprising
   (i) capsaicin in an amount of about 8% by weight,
   (ii) at least one amine-compatible polysiloxane in an amount of from 60 to 90% by weight,
   (iii) diethylene glycol monoethyl ether in an amount of from 10 to 25% by weight,
   (iv) ethyl cellulose in an amount of from 0 to 2% by weight, and
   (v) silicone oil in amount of from 0 to 5% by weight;
and
C) a skin contact layer;
   wherein the skin contact layer is an adhesive layer comprising a silicone gel adhesive, and wherein the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.

According to certain embodiments of the invention, the medical patch according to the invention is for use in a method of treating neuropathic pain, in particular chronic neuropathic pain, and preferably for use in a method of treating peripheral neuropathic pain, neuropathic pain associated with postherpetic neuralgia or diabetic peripheral neuropathy (DPN) of the hands and feet, post-surgical neuropathic pain, joint pain, or cancer pain.

According to certain embodiments of the invention, the present invention relates to the use of a medical patch according to the invention for the manufacture of a medicament for treating neuropathic pain, in particular chronic neuropathic pain, and preferably for the manufacture of a medicament for treating peripheral neuropathic pain, neuropathic pain associated with postherpetic neuralgia or diabetic peripheral neuropathy (DPN) of the hands or feet, post-surgical neuropathic pain, joint pain, or cancer pain.

According to certain embodiments of the invention, the invention relates to a method of treating neuropathic pain, in particular chronic neuropathic pain, and preferably peripheral neuropathic pain, neuropathic pain associated with postherpetic neuralgia or diabetic peripheral neuropathy (DPN) of the hands or feet, post-surgical neuropathic pain, joint pain, or cancer pain including applying a medical patch according to the invention to the skin of a patient.

### Definitions

Within the meaning of this invention, the term “medical patch” refers to a dermal delivery system by which the active agent (capsaicin) is administered to a patient and refers to the entire individual dosing unit that is applied, after removing an optionally present release liner, to the skin of the patient, and which comprises a therapeutically effective amount of capsaicin in an active agent-containing layer structure. The active agent-containing layer structure may be located on a release liner (a detachable protective layer), thus, the medical patch may further comprise a release liner.

In this context, the term “medical patch” is understood to mean an adhesive patch which can be a topical medical patch or a transdermal therapeutic system (TTS). Even if the topical medical patch as well as the TTS are topically applied in the sense that they are attached to the skin of the patient, the term “topical” or “topical administration” refers to the administration of capsaicin relying on passive diffusion into the skin itself, which creates a local effect at a point of action. In contrast, the term “TTS” refers to a system by which capsaicin is administered to the systemic circulation via transdermal delivery. Topical capsaicin administration may be preferable to capsaicin transdermal delivery systems regarding systemic side effects, drug interactions, contraindications and development of tolerance.

Within the meaning of this invention, the term “active agent-containing layer structure” refers to the capsaicin-containing structure providing the area of release for capsaicin during administration. The active agent-containing layer structure comprises a backing layer, an active agent-containing layer comprising capsaicin, optionally a membrane, and a skin contact layer as described herein. The active agent-containing layer structure thus comprises a therapeutically effective amount of capsaicin. According to particular embodiments, the active agent-containing layer structure is an active agent-containing self-adhesive layer structure.

Within the meaning of this invention, the term “therapeutically effective amount” refers to a quantity of capsaicin in the medical patch sufficient to provide, if administered by the medical patch to a patient, the desired therapeutic effect such as pain relief/reduction. A TTS usually contains more capsaicin in the system than is in fact provided to the skin and the systemic circulation, which is usually necessary to provide enough driving force for the delivery from the TTS to the systemic circulation.

Within the meaning of this invention, the terms “active agent” as well as “capsaicin” refer to capsaicin in any pharmaceutically acceptable chemical and morphological form and physical state. These forms include without limitation capsaicin in its free form, cocrystals, solvates, hydrates, clathrates, complexes and so on, as well as capsaicin in the form of particles, which may be micronized, crystalline and/or amorphous, and any mixtures of the aforementioned forms.

Capsaicin, where contained in a medium such as a solvent, may be dissolved or dispersed or in part dissolved and in part dispersed.

When the capsaicin is mentioned to be used in a particular form in the manufacture of the medical patch, this does not exclude interactions between this form of the capsaicin and other ingredients of the active agent-containing layer structure, e.g. salt formation or complexation, in the final medical patch. This means that, even if the capsaicin is included in one specific form, it may be present in the final medical patch in another form. Unless otherwise indicated, in particular the amount of the capsaicin in the layer structure relates to the amount of the capsaicin included in the medical patch during manufacture of the medical patch and is calculated based on capsaicin itself, but not on other forms thereof. The capsaicin starting material included in the medical patch during manufacture of the medical patch may be in the form of particles. Capsaicin may e.g. be present in the active agent-containing layer structure in the form of particles and/or dissolved.

In this context, the term “particles” refers to a solid, particulate material comprising individual particles, the dimensions of which are negligible compared to the material. In particular, the particles are solid, including plastic/deformable solids, including amorphous and crystalline materials. The term “dispersing” refers to a step or a combination of steps wherein a starting material (e.g. capsaicin) is not totally dissolved. Dispersing in the sense of the invention comprises the dissolution of a part of the starting material (e.g. capsaicin particles), depending on the solubility of the starting material (e.g. the solubility of capsaicin in the coating composition).

There are two main types of medical patches using (passive) active agent delivery, i.e. matrix-type medical patches and reservoir-type medical patches. The release of the active agent in a matrix-type medical patch is mainly controlled by the matrix including the active agent itself. In contrast thereto, a reservoir-type medical patch typically needs a rate-controlling membrane controlling the release of the active agent. In principle, also a matrix-type medical patch may contain a rate-controlling membrane. However, matrix-type medical patches are advantageous in that, compared to reservoir-type medical patches, usually no rate determining membranes are necessary and no dose dumping can occur due to membrane rupture. In summary, matrix-type medical patches are less complex in manufacture and easy and convenient to be used.

In this context, a “matrix-type medical patch” is understood to mean a system or structure wherein capsaicin is homogeneously dissolved and/or dispersed within a polymeric carrier, i.e. the matrix, which forms with capsaicin and optionally remaining ingredients a matrix layer. In such a system, the matrix layer controls the release of capsaicin from the medical patch. Preferably, the matrix layer has sufficient cohesion to be self-supporting so that no sealing between other layers is required. Accordingly, the active agent-containing layer may be an active agent-containing matrix layer, wherein capsaicin is homogeneously distributed within a polymer matrix. The active agent-containing matrix layer may comprise two active agent-containing matrix layers, which may be laminated together. Matrix-type medical patches may in particular be in the form of a “drug-in-adhesive”-type medical patch referring to a system wherein capsaicin is homogeneously dissolved and/or dispersed within a pressure sensitive adhesive matrix. In this connection, the active agent-containing matrix layer may also be an active agent-containing pressure sensitive adhesive layer or active agent-containing pressure sensitive adhesive matrix layer. A medical patch comprising capsaicin dissolved and/or dispersed within a polymeric gel, e.g. a hydrogel, is also considered to be of matrix-type in accordance with present invention.

Medical patches with a liquid active agent-containing reservoir are referred to by the term “reservoir-type medical patch”. In such a system, the release of capsaicin is preferably controlled by a rate-controlling membrane. In particular, the reservoir is sealed between the backing layer and the rate-controlling membrane. Accordingly, the active agent-containing layer may be an active agent-containing reservoir layer, which preferably comprises a liquid reservoir comprising capsaicin, and wherein the active agent-containing reservoir layer and the skin contact layer may be separated by the rate-controlling membrane. In the active agent-containing reservoir layer, capsaicin is preferably dissolved in a solvent such as ethanol or water or in silicone oil.

Reservoir-type medical patches are not to be understood as being of matrix-type within the meaning of the invention. However, microreservoir-type medical patches (biphasic systems having deposits (e.g. spheres, droplets) of an inner active agent-containing phase dispersed in an outer polymer phase), considered in the art to be a mixed form of a matrix-type medical patch and a reservoir-type medical patch that differ from a homogeneous single phase matrix-type medical patch and a reservoir-type medical patch in the concept of drug transport and drug delivery, are considered to be of matrix-type within the meaning of the present invention.

Thus, a microreservoir-type medical patch refers to a microreservoir systems, in which a liquid capsaicin preparation is dispersed in an adhesive matrix in the form of small droplets (“microreservoirs”). The size of the resulting droplets depends on the stirring conditions and the applied shear forces during stirring. It can be determined by an optical microscopic measurement (for example by Leica MZ16 including a camera, for example Leica DSC320) by taking pictures of the microreservoirs at different positions at an enhancement factor between 10 and 400 times, depending on the required limit of detection. By using imaging analysis software, the sizes of the microreservoirs can be determined. Microreservoirs systems are disclosed in U.S. Pat. Nos. 3,946,106, 4,053,580, 4,814,184 and 5,145,682, each of which is incorporated herein by reference. Specific microreservoirs systems are described in international patent publication WO0101967 the disclosure of which is incorporated herein by reference. These microreservoir systems contain, as base polymer, polysiloxanes and amphiphilic solvents for the microreservoir droplets.

Within the meaning of this invention, the term “active agent-containing layer” refers to a layer containing capsaicin and providing the area of release. The term covers active agent-containing reservoir layers and active agent-containing matrix layers, and in particular active agent-containing microreservoir layers. If the active agent-containing layer is an active agent-containing matrix layer, said layer is present in a matrix-type medical patch. As used herein, the active agent-containing layer is preferably an active agent-containing matrix layer, and it is referred to the final solidified layer, e.g., obtained after coating and drying a solvent-containing coating composition as described herein. Alternatively, an active agent-containing matrix layer is obtained after melt-coating and cooling. The active agent-containing matrix layer may also be manufactured by laminating two or more such solidified layers (e.g. dried or cooled layers) of the same composition to provide the desired area weight. According to particular embodiments, the matrix layer is a pressure sensitive adhesive matrix layer.

Within the meaning of this invention, the term “skin contact layer” refers to the layer included in the active agent-containing layer structure to be in direct contact with the skin of the patient during administration. The other layers of the active agent-containing layer structure do not contact the skin and do not necessarily have self-adhesive properties. The skin contact layer is directly attached to the active agent-containing layer, or a membrane is located between the active agent-containing layer and the skin contact layer. In this context, the term “membrane” is understood to mean a layer, which is provided between the active agent-containing layer and the skin contact layer and is at least semipermeable for capsaicin. The membrane may be a microporous film or a nonporous partition membrane. Preferred membranes can be selected from the group consisting of polyethylene membranes, polyurethane coated polyethylene terephthalate/polyethylene membranes, polyurethane membranes, and ethylene vinyl acetate membranes. According to the present invention, the skin contact layer is present as adhesive layer.

The sizes of the skin contact layer and the active agent-containing layer or the sizes of the skin contact layer, the membrane and the active agent-containing layer are usually coextensive and correspond to the area of release. However, the area of the skin contact layer, as well as optionally the membrane, may also be greater than the area of the active agent-containing layer. In such a case, the area of release still refers to the area of the active agent-containing layer.

Within the meaning of the invention, the term “backing layer” refers to a layer which supports the active agent-containing layer. At least one backing layer in the medical patch and usually the backing layer of the active agent-containing layer is substantially impermeable to capsaicin, as well as optionally any additive, contained in the layer during the period of storage and administration and thus prevents active loss or cross-contamination in accordance with regulatory requirements. According to particular embodiments, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyesters, polyurethanes, and mixtures thereof. Suitable backing layers may be siliconized in order to improve the adhesion of the active agent-containing layer to the backing layer.

Furthermore, an adhesive overlay may be present. In this context, the term “adhesive overlay” is understood to mean a self-adhesive layer structure that is free of capsaicin and larger in area than the active agent-containing structure and provides additional area adhering to the skin, but no area of release of capsaicin. It enhances thereby the overall adhesive properties of the medical patch. The area of said adhesive overlay adds to the overall size of the medical patch but does not add to the area of release. The adhesive overlay may comprise a self-adhesive polymer or a self-adhesive polymer mixture selected from the group of acrylic polymers, polyisobutylenes, styrene-isoprene-styrene copolymers, polysiloxanes, and mixtures thereof, which may be identical to or different from any polymer or polymer mixture included in the active agent-containing self-adhesive layer structure. The adhesive overlay comprises a backing layer that may provide occlusive or non-occlusive properties and an adhesive layer. According to particular embodiments, the backing layer of the adhesive overlay provides non-occlusive properties.

Within the meaning of this invention, the term “area weight” refers to the dry weight of a specific layer, e.g. of the active agent-containing layer, provided in g/m². The area weight values are subject to a tolerance of ±10%, preferably +7.5%, due to manufacturing variability.

If not indicated otherwise “%” refers to wt. % (% by weight).

Within the meaning of this invention, the term “polymer”, e.g., polymer I or II, refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, above 5000, or above 10,000 Dalton. Correspondingly, compounds with a molecular weight below 2000, below 5000, or below 10,000 Dalton are usually referred to as oligomers.

Within the meaning of this invention, the term “pressure sensitive adhesive” refers to a material that in particular adheres with finger pressure, is permanently tacky, exerts a strong holding force and should be removable from smooth surfaces without leaving a residue. It is obtainable from a solvent-containing adhesive coating composition after coating on a film and evaporating the solvents (e.g. n-heptane or ethyl acetate). In this context, the term “solvent” is understood to mean any liquid substance, which preferably is a volatile organic liquid such as methanol, ethanol, isopropanol, acetone, ethyl acetate, methylene chloride, hexane, n-heptane, toluene, and mixtures thereof.

Within the meaning of the invention, the term “silicone-acrylic hybrid polymers” refers to a hybrid polymer based on silicones and acrylates in the form of a pressure-sensitive adhesive. Silicone acrylic hybrid pressure-sensitive adhesives are described, for example, in EP 2 599 847 and WO 2016/130408. It was found that, depending on the solvent in which the silicone acrylic hybrid PSA is supplied, the arrangement of the silicone phase and the acrylic phase providing a silicone or acrylic continuous external phase and a corresponding discontinuous internal phase is different. If the silicone acrylic hybrid PSA is supplied in n-heptane, the composition contains a continuous, silicone external phase and a discontinuous, acrylic internal phase. If the silicone acrylic hybrid PSA composition is supplied in ethyl acetate, the composition contains a continuous, acrylic external phase and a discontinuous, silicone internal phase.

Within the meaning of the invention, the term “silicone-based polymer” refers to a non-hybrid polymer (i.e. a polymer, which does not include a hybrid species) comprising polysiloxanes. Polysiloxanes can be made from solvent-free two-component systems or a solution in organic solvents. They exist in two fundamentally different variants: polysiloxanes which have free silanol groups and amine resistant polysiloxanes which are distinguished in that the free silanol groups are derivatized by trimethylsilyl groups. The methyl groups can be completely or partially replaced by other alkyl radicals or alternatively phenyl radicals. Polysiloxanes as used herein are synthesized from linear bifunctional and branched polyfunctional oligomers, the ratio of which determines the physical properties thereof. More polyfunctional oligomers result in a more cross-linked adhesive with a higher cohesion and a reduced tack, less polyfunctional oligomers result in a higher tack and a reduced cohesion. It is preferred for the silicone-based polymer to be a mixture of high tack and medium tack, or high tack and low tack, polysiloxanes. According to particular embodiments, the at least one silicone-based polymer is a silicone-based pressure sensitive adhesive.

Within the meaning of this invention, the term “silicone gel adhesive” refers to an elastic, jelly-like material formed by lightly crosslinking silicone polymers. It may be prepared from a gel producing composition as described further below upon curing. In particular, the silicone gel adhesive forms upon curing of polysiloxanes comprising reactive groups such as Si—H reactive groups and aliphatic unsaturated groups, which react with each other in the presence of a hydrosilylation catalyst. According to particular embodiments, the silicone gel adhesive is based on a polydimethylsiloxane network, which may be formed in an addition reaction (hydrosilylation) between vinyl function polydimethylsiloxane groups (polymer) and hydrogen functional siloxanes (cross-linker). Accordingly, the silicone gel adhesive is typically applied by using a curable gel producing (2-component) composition, which solidifies upon curing.

Within the meaning of the invention, the term “natural or synthetic rubbers” refers to an elastomer which is obtainable by polymerizing an unsaturated hydrocarbon, such as isoprene (2-methyl-1,3-butadiene), or by copolymerizing such hydrocarbons with styrene, butadiene, or the like. It includes natural and synthetic polyisoprene, polybutylene and polyisobutylene, styrene/butadiene polymers, styrene-isoprene-styrene block copolymers, hydrocarbon polymers such as butyl rubber, halogen-containing polymers such as polyacrylic-nitrile, polytetrafluoroethylene, polyvinylchloride, polyvinylidene chloride, and polychlorodiene, as well as other copolymers thereof. In certain embodiments, natural or synthetic rubbers may be styrenic triblock copolymers or polyisobutylenes.

Within the meaning of this invention, the term “saturation concentration” refers to that active agent concentration corresponding to the equilibrium state in which the solvent (i.e. the polymer II of the skin contact layer) cannot dissolve further solute (i.e. the capsaicin), and as a result the solid solute is present in equilibrium with the solid solution at defined temperature (room temperature—unmodified temperature found indoors in the laboratory where experiments are conducted and usually lies within 15 to 35° C., or about 18 to 25° C.). The saturation concentration of the active agent can be indicated in % by weight, based on the total weight of the active agent layer or skin contact layer, respectively. The saturation concentration can be determined e.g. using a method described by Liu, P., Gargiulo, P., Wong, J., and Novartis. Pharm. Research. Vol. 14, p. 317 (1997), herein referred to as “Sandwich method”, in which a multi-layered laminate is prepared comprising an upper and lower protective layer sandwiching a donor layer and an acceptor layer separated by a partitioning membrane that is permeable to the active agent. Since the donor layer contains an excess of the active agent and the acceptor layer is substantially free of the active agent, the active agent diffuses out of the donor layer through the partitioning membrane into the acceptor layer until the saturation concentration is achieved. The donor layer and the acceptor layer are manufactured from the respective polymer II of the skin contact layer. The donor layer is oversaturated with the active agent, while the acceptor layer is prepared analogously to the donor layer but does not comprise the active agent. The sandwich systems prepared are stored for a certain time, e.g., 7 days, at room temperature, to allow the active agent to diffuse from the donor layer into the acceptor layer. Then, the remaining active agent concentration of the donor layer is determined by means of HPLC (high performance liquid chromatography) in order to finally obtain the saturation concentration of the active agent in the respective polymer II of the skin contact layer.

As used herein, solubility parameters (SPs) are defined as the sum of all the intermolecular attractive forces, which, as a numerical estimate, are empirically related to the extent of mutual solubility of chemical species. The most convenient method to determine solubility parameters is Hildebrand's method, which computes the solubility parameter from molecular weight, boiling point and density data, which are commonly available for many materials: SP=(ΔEᵥ/V)^{1/2}, where V=molecular weight/density and ΔEᵥ=energy of vaporization. For materials, such as high molecular weight polymers, which have vapor pressures too low to detect, several methods have been developed which use the summation of atomic and group contributions to vaporization. Such a method of calculating the solubility parameter of a material has been described e.g. by Small, J. Applied Chem. Vol. 3, p. 71 (1953). Some solubility parameters (calculated by Small's method) of exemplary polymers useful in the practice of the invention are as follows: Polydimethylsiloxane 14.9 Mpa^{1/2}, polyisobutylene 15.7 Mpa^{1/2}, polyethylene/butylene 16.2 MPa^{1/2}, polyisoprene 16.6 Mpa^{1/2}, polyethylene 16.6 MPa^{1/2}, polybutadiene 16.6 Mpa^{1/2}, polybutadiene-co-styrene (75/25 to 72/28) 17.4 MPa^{1/2}, polystyrene 18.6 Mpa^{1/2}, polymethyl methacrylate 19.0 MPa^{1/2}, polymethyl acrylate 19.8 Mpa^{1/2}.

Within the meaning of the invention, the term “soluble polyvinylpyrrolidone” refers to polyvinylpyrrolidone, also known as povidone, which is soluble with more than 10% in at least ethanol, preferably also in water, diethylene glycol, methanol, n-propanol, 2 propanol, n-butanol, chloroform, methylene chloride, 2-pyrrolidone, macrogol 400, 1,2 propylene glycol, 1,4 butanediol, glycerol, triethanolamine, propionic acid and acetic acid. Examples of polyvinylpyrrolidones which are commercially available include Kollidon® 12 PF, Kollidon® 17 PF, Kollidon® 25, Kollidon® 30 and Kollidon® 90 F supplied by BASF, or povidone K90F. The different grades of Kollidon® are defined in terms of the K-Value reflecting the average molecular weight of the polyvinyl-pyrrolidone grades. Kollidon® 12 PF is characterized by a K-Value range of 10.2 to 13.8, corresponding to a nominal K-Value of 12. Kollidon® 17 PF is characterized by a K-Value range of 15.3 to 18.4, corresponding to a nominal K-Value of 17. Kollidon® 25 is characterized by a K-Value range of 22.5 to 27.0, corresponding to a nominal K-Value of 25, Kollidon® 30 is characterized by a K-Value range of 27.0 to 32.4, corresponding to a nominal K-Value of 30. Kollidon® 90 F is characterized by a K-Value range of 81.0 to 97.2, corresponding to a nominal K-Value of 90. Preferred Kollidon® grades are Kollidon® 12 PF, Kollidon® 30 and Kollidon® 90 F. In this contex, the term “K-Value” refers to a value calculated from the relative viscosity of polyvinylpyrrolidone in water according to the European Pharmacopoeia (Ph.Eur.) and USP monographs for “Povidone”. For all grades and types of polyvinylpyrrolidone, it is preferred that the amount of peroxides is within certain limits, in particular, the peroxide amount is equal to or less than 500 ppm, equal to or less than 150 ppm, or equal to or less than 100 ppm.

The medical patch according to the present invention can be characterized by certain parameters as measured in an in vitro skin permeation test.

The in vitro permeation test can be performed with human or animal skin and preferably with dermatomed split-thickness human skin with a thickness of 500 μm and an intact epidermis, and with 0.9% sodium chloride solution as receptor medium (32° C. with 0.1% saline azide) with or without addition of a maximum of 40 vol-% organic solvent e.g. ethanol, acetonitrile, isopropanol, dipropylenglycol, PEG 400 so that a receptor medium may e.g. contain 60 vol-% of 0.9% sodium chloride, 30 vol-% dipropylenglycol and 10 vol-% acetonitrile.

Where not otherwise indicated, the in vitro permeation test is performed in a Franz diffusion cell with heat separated human epidermis, and with 0.9% sodium chloride solution as receptor medium (32° C. with 0.1% saline azide). The amount of capsaicin permeated into the receptor medium is determined in regular intervals using a partial validated HPLC method (column: stainless steel column 150 mm×4.6 mm internal diameter with C18 base and acid deactivated stationary phase, 3.5 μm particle size, e.g. Zorbax SB C18 (Agilent); column temperature: 25° C.; mobile phase: acetonitrile/water/TEA=20:80:0.35 (v/v/v) with a UV photometric detector by taking a sample volume. The receptor medium is completely or in part replaced by fresh medium when taking the sample volume, and the measured amount of capsaicin permeated relates to the amount permeated between the two last sampling points and not the total amount permeated so far.

Thus, within the meaning of this invention, the parameter “permeated amount” is provided in μg/cm² and relates to the amount of capsaicin permeated in a sample interval at certain elapsed time. E.g., in an in vitro permeation test as described above, wherein the amount of capsaicin permeated into the receptor medium has been e.g. measured at minutes 0, 30, 60, 90, 120, 180 and 240, the “permeated amount” of capsaicin can be given e.g. for the sample interval from minute 90 to minute 120 and corresponds to the measurement at minute 120, wherein the receptor medium has been exchanged completely at minute 90. The permeated amount can also be given as a “cumulative permeated amount”, corresponding to the cumulated amount of capsaicin permeated at a certain point in time. E.g., in an in vitro permeation test as described above, wherein the amount of capsaicin permeated into the receptor medium has been e.g. measured at minutes 0, 30, 60, 90, 120, 180 and 240, the “cumulative permeated amount” of capsaicin at minute 120 corresponds to the sum of the permeated amounts from minute 0 to minute 30, minute 30 to minute 60, minute 60 to minute 90 and minute 90 to minute 120.

Within the meaning of this invention, the parameter “skin permeation rate”, also referred to as “A flux rate”, for a certain sample interval at certain elapsed time is provided in μg/(cm²*min) and is calculated from the permeated amount in said sample interval as measured by in vitro permeation test as described above in μg/cm², divided by the minutes of said sample interval. E.g. the skin permeation rate in an in vitro permeation test as described above, wherein the amount of capsaicin permeated into the receptor medium has been e.g. measured at minutes 0, 30, 60, 90, 120, 180 and 240, the “skin permeation rate” at Δtime point 105 is calculated as the permeated amount in the sample interval from minute 90 to minute 120 divided by 30 minutes. A “cumulative skin permeation rate” can be calculated from the respective cumulative permeated amount by dividing the cumulative permeated amount by the elapsed time.

Within the meaning of this invention, the above parameters “permeated amount” and “skin permeation rate” (as well as “cumulative permeated amount” and “cumulative skin permeation rate”) refer to mean values calculated from at least 6 in vitro permeation test experiments. Where not otherwise indicated, the standard deviation (SD) of these mean values refer to a corrected sample standard deviation, calculated using the formula:

$$SD = \sqrt{\frac{1}{n - 1} {\sum }_{i = 1}^{n} {( {x}_{i} - \overline{x} )}^{2}}$$

wherein n is the sample size, {x₁, x₂, . . . xₙ} are the observed values and x̅ is the mean value of the observed values.

Within the meaning of this invention, the term “room temperature” refers to the unmodified temperature found indoors in the laboratory where the experiments are conducted and usually lies within 15 to 35° C., or about 18 to 25° C.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated. Preferably, the patient suffers from neuropathic pain or mixed neuropathic and/or nociceptive pain such as joint pain or cancer pain.

Within the meaning of this invention, the term “neuropathic pain” refers to pain caused by a lesion or disease of the somatosensory nervous system. In this context, the term “chronic neuropathic pain” is understood to mean neuropathic pain lasting for at least three months. When suffering from neuropathic pain, most patients complain of an ongoing or intermittent spontaneous pain of, e.g., burning, pricking, squeezing quality, which may be accompanied by evoked pain, particular to light touch and cold. Ectopic activity in, e.g., nerve-end neuroma, compressed nerves or nerve roots, dorsal root ganglia, and the thalamus may in different conditions underlie the spontaneous pain. Neuropathic pain includes peripheral neuropathic pain that particularly affects the peripheral nerves, meaning the nerves located outside the brain and spinal cord. In particular, neuropathic pain within the meaning of this invention relates to post-surgical neuropathic pain, as well as neuropathic pain associated with postherpetic neuralgia or diabetic peripheral neuropathy of the hands or feet.

In this context, the term “post-surgical neuropathic pain” is understood to mean chronic pain that develops after a surgical procedure and persists beyond the healing process, i.e. at least three months after the surgery. The pain is either localized to the surgical field or area of injury, projected to the innervation territory of a nerve situated in this area, or referred to a dermatome (after surgery/injury to deep somatic or visceral tissues). Chronic post-surgical pain is the result of nerve damage and can be due to the surgery itself or other causes of pain including infection, malignancy, etc.

In this context, the term “postherpetic neuralgia”, also referred to as post-shingles nerve pain, is understood to mean pain occurring if nerves are damaged due to a previous herpes zoster infection, commonly referred to as shingles. Symptoms of post-shingles nerve pain are often limited or localized to the area of the skin where the shingles outbreak first occurred, in the band around the trunk, usually on one side of the body. Less common symptoms of post-shingles nerve pain include itching, numbness, or feeling “pins and needles.”

In this context, the term “diabetic peripheral neuropathy”, also referred to as diabetic nerve pain”, is understood to mean pain occurring if nerves are damaged as a result of diabetes. Although diabetic nerve pain can affect any nerve, it is most often felt in extremities, such as the hands or feet.

Within the meaning of this invention, the term “joint pain” refers to an articular condition, such as discomfort, aches or soreness in any of the body's joints of a patient, including spine, shoulders, hips, elbows and knees. This includes in particular joint pain caused by arthritis, such as osteoarthritis. In this context, the term “osteoarthritis” is understood to mean a degenerative disease characterized by cartilage erosion, bony hypertrophy, subchondral sclerosis, and synovial and capsular changes. It is clinically characterized by joint pain, stiffness, and functional limitation. Although the pain of osteoarthritis is traditionally considered to be nociceptive, some patients also have neuropathic pain. The joint pain may be in particular knee pain, elbow pain, hip pain, shoulder pain, pain of the hands or feet, or pain of the (lower) back.

Within the meaning of this invention, the term “cancer pain” relates to neuropathic cancer pain caused by nerve damage attributable to the cancer per se, and/or treatments including chemotherapy, radiotherapy, and surgery. Cancer pain caused by the tumor per se usually involves both nociceptive and neuropathic components, and mixed pain is more common than neuropathic cancer pain caused by cancer treatments. Most cancer pain caused by chemotherapy is purely neuropathic in nature. Neuropathic cancer pain is nerve-related (typically neuron-related) pain characterized as a burning or electrical sensation; however, it sometimes manifests as decreased sensation or actual muscle weakness.

According to the invention, the medical patch as described herein is suitable for use in a method of treatment, in which the medical patch is applied for a short period of time (application time), but provides for the desired effect of pain relief for an extended period of time (effect time). Within the application time, it is preferred for the medical patch to release almost the total amount of capsaicin contained in the active agent-containing layer of the medical patch. The capsaicin is believed to be able to desensitize and defunctionalize the TRPV1 receptor within the application time, so that it can provide sustained pain relief that lasts during the effect time.

Within the meaning of this invention, the term “short period of time”, i.e., the application time, relates to a period of less than or about 240 minutes, less than or about 180 minutes, less than or about 120 minutes, less than or about 90 minutes, less than or about 60 minutes, less than or about 45 minutes, less than or about 30 minutes, less than or about 15 minutes, or 30 to 90 minutes.

Within the meaning of this invention, the term “extended period of time”, i.e., the effect time relates to a period of at least or about 1 week, at least or about 2 weeks, at least or about 1 month, at least or about 1.5 months, at least or about 2 months, at least or about 3 month, or 1 to 3 months.

The interval between two dosage form administrations, also called dosing interval, needs to be adapted accordingly. Within the meaning of the present invention, the term “dosing interval” refers to the period of time between two consecutive medical patch administrations, i.e. the interval between two consecutive points in time a medical patch is applied to the skin of the patient. Once applied, the medical patch is only maintained on the skin of the patient for the application time and subsequently removed. However, the dosing interval lasts until a new medical patch is applied to the skin.

Within the meaning of this invention, the term “coating composition” refers to a composition comprising all components of the active agent-containing layer or the skin contact layer, respectively, which may be coated onto the backing layer or release liner to form the active agent-containing layer and the skin contact layer upon drying.

Within the meaning of this invention, the term “dissolve” refers to the process of obtaining a solution, which is clear and does not contain any particles, as visible to the naked eye.

Within the meaning of this invention, the term “cross-linking” refers to the process of cross-link functional groups contained within the active-free coating composition.

Within the meaning of this invention, and unless otherwise specified, the term “about” refers to an amount that is ±10% of the disclosed amount. In some embodiments, the term “about” refers to an amount that is ±5% of the disclosed amount. In some embodiments, the term “about” refers to an amount that is ±2% of the disclosed amount.

### BRIEF DESCRIPTION OF THE DRAWINGS

FIG. 1a depicts the capsaicin cumulative permeated amount of medical patches prepared according to Examples 1 as well as the Reference Example.

FIG. 1b depicts the capsaicin skin permeation rate of medical patches prepared according to Examples 1 as well as the Reference Example.

### DETAILED DESCRIPTION

### Medical Patch Structure

The present invention relates to a medical patch for the administration of capsaicin. The medical patch can be either a topical medical patch or a transdermal therapeutic system. According to certain embodiments, the patch is a topical medical patch, in particular for the topical administration of capsaicin.

The medical patch according to the present invention comprises an active agent-containing layer structure, said active agent-containing layer structure comprising:
A) a backing layer;
B) an active agent-containing layer comprising
   (i) capsaicin, and
   (ii) at least one silicone-based polymer;
and
C) a skin contact layer;
   wherein the skin contact layer is an adhesive layer comprising a polymer II (the at least one silicone-based polymer in the active agent-containing layer is also referred to as polymer I). The active agent capsaicin is sparingly soluble in the skin contact layer, e.g., the saturation concentration of the capsaicin in the skin contact layer may be less than 0.1% by weight. In particular embodiments, the medical patch according to the present invention comprises an active agent-containing layer structure, said active agent-containing layer structure comprising:
A) a backing layer;
B) an active agent-containing layer comprising
   (i) capsaicin, and
   (ii) at least one silicone-based polymer;
and
C) a skin contact layer;
   wherein the skin contact layer is an adhesive layer comprising a silicone gel adhesive.

In an embodiment, the active agent-containing layer structure may or may not comprise a membrane which is located between the active agent-containing layer and the skin contact layer. In such embodiment, the membrane preferably is a rate controlling membrane. However, for the specific application in the present case. i.e., having a short application time in which rapid release of active compound is desired, a rate controlling membrane is usually not present.

Preferably, the aforementioned layers of the medical patch according to the invention are directly attached to each other, i.e. the backing layer is directly attached to the active agent-containing layer, which is on the other side directly attached to the skin contact layer. Alternatively, the active agent-containing layer is directly attached to a membrane, which is on the other side directly attached to the skin contact layer. In other words, the medical patch according to the present invention comprises its layers in the following order: (1) backing layer, (2) active agent-containing layer, and (3) skin contact layer, or (1) backing layer, (2) active agent-containing layer, (3) membrane, and (4) skin contact layer.

The backing layer is in particular substantially impermeable to capsaicin. It may consist of a polyester film, preferably with a thickness of 10-20 μm, or of an ethylene-vinyl acetate copolymer.

In accordance with the invention, the medical patch comprises capsaicin, in particular in a therapeutically effective amount. Thus, in certain embodiments, the medical patch comprises the capsaicin in an amount of from 0.5 to 180 mg, from 1.2 to 90 mg, or 19 to 45 mg. In some embodiments, the medical patch comprises the capsaicin in an amount of about 179 mg. In other embodiments, the medical patch comprises the capsaicin in an amount of about 60 mg, of about 45 mg, of about 30 mg, of about 25 mg, of about 10 mg, or of about 1 mg.

The medical patch according to the present invention may be a matrix-type medical patch or a reservoir-type medical patch, and preferably is a matrix-type medical patch. In particular embodiments, the medical patch according to the present invention is a matrix-type medical patch, wherein the capsaicin is homogeneously dissolved and/or dispersed within a polymeric carrier, i.e. the matrix, which forms together with the capsaicin and optionally further additives a matrix layer. Accordingly, it is preferred that the active agent-containing layer is an active agent-containing matrix layer. Thus, in certain embodiments of the medical patch according to the present invention, the active agent-containing layer is an active agent-containing matrix layer comprising
(i) capsaicin, and
(ii) the at least one silicone-based polymer.

In particular embodiments, the medical patch according to the present invention is a microreservoir-type medical patch.

The active agent-containing layer structure according to the invention is normally located on a detachable protective layer (release liner) from which it is removed immediately before application to the surface of the patient's skin. Thus, the medical may further comprise a release liner. A medical patch protected this way is usually stored in a seam-sealed pouch. The packaging may be child resistant and/or senior friendly.

A medical patch according to one embodiment of the present invention, which comprises an adhesive skin contact layer comprising a silicone gel adhesive, when applied to a patient's skin, provides improved wearing properties as well as a clean and painless removal. If necessary, e.g. in the case of repositioning, the medical patch can be removed and applied again without loss of the adhesiveness. Thus, the medical patch is even suitable for problematic applications sites, such as, e.g., hand or foot. Such application sites are particularly challenging due to their uneven surface and complexity and need to be considered in view of shape and size of the active agent-containing layer structure.

Thus, in a specific embodiment, the active agent-containing layer structure has a hexagonal shape, wherein the backing layer, the active agent-containing layer and the skin contact layer are coextensive and provide the hexagonal shape of the active agent-containing layer structure and the hexagonal shape comprises at least one hexagon, wherein
all pairs of opposite sides of the hexagon are parallel, and
the sides of the hexagon have a length of from 1.5 to 10 cm.

In certain embodiments, the at least one hexagon may be at least one convex hexagon, and the hexagonal shape may preferably comprise one or two convex hexagons. In particular the hexagonal shape is a convex hexagon. In particular embodiments, the hexagonal shape is a double-hexagon formed of two identical convex hexagons sharing two adjacent vertices and their common side, wherein preferably said common side is perforated for easy tear-off.

### Active Agent-Containing Layer

As outlined in more detail above, the medical patch according to the present invention comprises an active agent-containing layer structure comprising inter alia an active agent-containing layer, which comprises
(i) capsaicin, and
(ii) at least one silicone-based polymer.

In a preferred embodiment, the active agent-containing layer contains at least 0.30 mg/cm², at least 0.50 mg/cm², or at least 0.60 mg/cm² capsaicin per area of release, and/or less than 1.0 mg/cm², less than 0.8 mg/cm², or less than 0.7 mg/cm² capsaicin per area of release. In particular, the active agent-containing layer contains from 0.30 mg/cm² to 1.0 mg/cm², from 0.30 mg/cm² to 8.0 mg/cm², from 0.50 mg/cm² to 8.0 mg/cm², from 0.60 mg/cm² to 0.8 mg/cm², or from 0.60 mg/cm² to 0.7 mg/cm² capsaicin per area of release.

According to certain embodiments, the active agent-containing layer comprises the capsaicin in an amount of from 2 to 20% by weight, from 5 to 15% by weight, or from 5 to 10% by weight. In particular embodiments, the active agent-containing layer comprises the capsaicin in an amount of about 8% by weight.

Further, according to certain embodiments, the at least one silicone-based polymer is present in the active agent-containing layer in an amount of from 20 to 90% by weight, or 60 to 90% by weight, based on the total weight of the active agent-containing layer. It is to be understood that the aforementioned weight percent amounts refer to the overall amount of the at least one silicone-based polymer. For example, if two silicone-based polymers are present, the overall amount in the active agent-containing layer is from 20 to 90% by weight, or 60 to 90% by weight, based on the total weight of the active agent-containing layer.

Thus, in certain embodiments of the medical patch according to the present invention, the active agent-containing layer comprises
(i) capsaicin in an amount of from 5 to 10% by weight, in particular an amount of about 8% by weight, and
(ii) at least one silicone-based polymer in amount of from 60 to 90% by weight.

The capsaicin is preferably homogeneously distributed within the active agent-containing layer, in particular the active agent-containing matrix layer. As used herein, an active agent-containing matrix layer is a layer containing the capsaicin dissolved or dispersed in the at least one silicone-based polymer, or containing capsaicin dissolved in a solvent to form a capsaicin-solvent mixture that is dispersed in the form of deposits (in particular droplets) in the at least one silicone-based polymer. In a particularly preferred medical patch, the active agent-containing layer comprises the capsaicin dissolved in an amphiphilic solvent (active agent-containing solution) as microreservoir droplets distributed within the active agent-containing matrix layer. The proportion of the microreservoir droplets in the active agent-containing matrix layer is usually less than about 40% by weight, or less than about 35% by weight, or between about 20% and about 30% by weight.

Suitable amphiphilic solvents include butanediols, in particular 1,3-butanediol, dipropylene glycol, tetrahydrofurfuryl alcohol, diethylene glycol dimethyl ether, diethylene glycol monoethyl ether (DGME), diethylene glycol monobutyl ether, propylene glycol, carboxylic acid esters of tri- and diethylene glycol, polyethoxylated fatty alcohols of 6-18 C atoms or 2,2-dimethyl-4-hydroxymethyl-1, 3-dioxolane, or any mixture thereof or mixtures of these solvent. In particular embodiments, the amphiphilic solvent is diethylene glycol monoethyl ether, also known by the trade name Transcutol®. As the solubility of capsaicin in DGME is about 50% by weight, the medical patch is not amenable to recrystallization of capsaicin, even under unfavorable conditions such as, e.g., the partial loss of the solvent or low temperature. In addition, DGME acts as a penetration enhancer, which is released along with the capsaicin and thus causes the concentration as well as the thermodynamic activity of the capsaicin in the microreservoir system, to remain at a high level despite release.

Thus, in certain embodiments of the present invention, the active agent-containing layer comprises diethylene glycol monoethyl ether, and preferably comprises diethylene glycol monoethyl ether in an amount of from 10 to 25% by weight.

In addition, it has been shown that the dispersion of active agent-containing solution can be realized more easily if the viscosity of the active agent-containing solution is increased by the addition of a suitable agent such as, e.g., a cellulose derivative, including ethylcellulose or hydroxypropyl-cellulose.

Thus, in certain embodiments of the present invention, the active agent-containing layer comprises a viscosity-increasing additive selected from the group consisting of cellulose derivatives and high molecular weight polyacrylic acids, and any mixture thereof. In particular, the active agent-containing layer comprises ethyl cellulose, and preferably comprises ethyl cellulose in an amount of from 0 to 2% by weight.

In certain embodiments of the medical patch according to the present invention, the active agent-containing layer comprises
(i) capsaicin in an amount of from 5 to 10% by weight, in particular an amount of about 8% by weight,
(ii) at least one silicone-based polymer in amount of from 60 to 90% by weight,
(iii) diethylene glycol monoethyl ether in an amount of from 10 to 25% by weight, and
(iv) ethyl cellulose in an amount of from 0 to 2% by weight.

According to certain embodiments, the at least one silicone-based polymer is a non-hybrid polymer comprising polysiloxanes. It is preferably a thermoplastic polymer, which is applied by hot-melt or a solvent-based process and typically does not undergo further curing to solidify.

Further details regarding these polymers are provided below. In particular embodiments, the at least one silicone-based polymer is at least one amine-compatible polysiloxane.

In certain embodiments, the active agent-containing layer comprises silicone oil, and preferably comprises silicone oil in an amount of from 0 to 5% by weight, in particular 0.5 to 5% by weight. This includes in particular dimethicone.

Thus, in one preferred embodiment of the medical patch according to the present invention, the active agent-containing layer comprises
(i) capsaicin in an amount of from 5 to 10% by weight, in particular an amount of about 8% by weight,
   (ii) at least one amine-compatible polysiloxane in amount of from 60 to 90% by weight, in particular 58 to 85% by weight,
   (iii) diethylene glycol monoethyl ether in an amount of from 10 to 25% by weight,
   (iv) ethyl cellulose in an amount of from 0 to 2% by weight, and
   (v) silicone oil in amount of from 0 to 5% by weight.

Further, in a preferred embodiment, the area weight of the active agent-containing layer ranges from 30 to 200 g/m², or from 50 to 120 g/m².

### Skin Contact Layer

As outlined in more detail above, the medical patch according to the present invention comprises an active agent-containing layer structure comprising inter alia a skin contact layer, wherein the skin contact layer is an adhesive layer comprising a polymer II. The skin contact layer is preferably directly attached to the active agent-containing layer.

The medical patch according to the present invention is in particular characterized by the low solubility of the capsaicin in the skin contact layer, and in certain embodiments, the saturation concentration of the capsaicin in the skin contact layer is less than 0.1% by weight, preferably as determined by the “sandwich method”. In certain embodiments, the saturation concentration of the capsaicin in the skin contact layer is less than 0.05% by weight, less than 0.02% by weight, or less than 0.01% by weight. Preferably, the saturation concentration of the capsaicin in the skin contact layer is about 0% by weight. The saturation concentration relates to the amount of capsaicin being present in the skin contact layer, based on the total weight of the skin contact layer.

In certain embodiments, the saturation concentration of the capsaicin in the skin contact layer is less than a concentration of the capsaicin resulting in any unintended adverse effect such as a skin irritation upon contact after a short period of time. Such a concentration may be determined empirically by in vivo testing, by observing whether an adverse effect such as any form of skin irritation (redness, erythema, itching or other skin reaction) occurs or does not occur after applying model adhesive layers with defined capsaicin concentrations on the skin for a short period of time, e.g., 5 seconds, 10 seconds, 30 seconds or 1 minute. In particular, different model layers representing a series of capsaicin concentrations may be tested to determine the highest acceptable saturation concentration which will not yet cause any unintended adverse effect such as a skin irritation reaction. On the other hand, whether or not a medical patch with a certain set of capsaicin and skin contact layer results in a saturation concentration not resulting in any adverse effect can be simply determined (without a series of different concentration) by testing a model adhesive layer which is saturated with the capsaicin, or said medical patch, i.e., by applying on the skin as outlined above.

In accordance with the invention, the skin contact layer shields the capsaicin contained in the active agent-containing layer from the skin of the patient or other applying/removing person before and/or after application of the medical patch. The skin contact layer thus needs to be substantially free of the capsaicin. This means that the skin contact layer is typically manufactured as a layer free of the capsaicin. However, due to the concentration gradient, the capsaicin usually may migrate from the active agent-containing layer to the skin contact layer over time, until an equilibrium is reached. This migration is, however, limited by the saturation concentration of the capsaicin in the skin contact layer. In certain embodiments, the skin contact layer does not allow the capsaicin to be present at a concentration of more than 0.1% by weight.

Thus, in certain embodiments, the skin contact layer comprises the capsaicin in an amount of less than 0.1% by weight, based on the total weight of the skin contact layer. In particular embodiments, the skin contact layer comprises the capsaicin in an amount of less than 0.01% by weight, based on the total weight of the skin contact layer.

The polymer II in the skin contact layer is decisive for the adhesive properties as well as the reduction of skin irritation inter alia due to its resiliency. Furthermore, the polymer II does not negatively affect the delivery of capsaicin. In certain embodiments the skin contact layer comprises the polymer II in an amount of at least 95% by weight, at least 99% by weight, or in an amount of about 100% by weight, based on the total weight of the skin contact layer. In particular, the skin contact layer may essentially consist of the polymer II. It is to be understood that the aforementioned weight percent amounts refer to the overall amount of the polymer II. For example, if the polymer II is a mixture of polymers, the overall amount in the skin contact layer is from 50 to 100% by weight, based on the total weight of the skin contact layer.

Polymers which are suitable as the polymer II in accordance with the invention are in particular polymers allowing the capsaicin to concentrate to not more than 0.1% by weight, not more than 0.05% by weight, not more than 0.02% by weight, or not more than 0.01% by weight, i.e. polymers in which the capsaicin is substantially insoluble. Thus, according to certain embodiments, the polymer II may be a polymer or a mixture of polymers in which the capsaicin is substantially insoluble.

Accordingly, the solubility parameter of the polymer II may differ from, in particular may be lower than the solubility parameter of the capsaicin by at least 5.0 MPa^{1/2}, at least 6.0 MPa^{1/2} at least 8.0 MPa/2, or at least 10.0 MPa^{1/2}. In particular, the solubility parameter of the polymer II may be less than 18.5 MPa^{1/2} less than 18.0 MPa^{1/2} less than 17.5 MPa^{1/2} less than 17.0 MPa^{1/2} less than 16.0 MPa^{1/2}, or less than 15.0 MPa^{1/2}, preferably as calculated by Small's method.

The polymer II may be selected from pressure sensitive adhesive polymers. Thus, in certain embodiments, the polymer II may be a pressure sensitive adhesive or a mixture of pressure sensitive adhesives.

In certain embodiments, the polymer II may be a polymer or a mixture of polymers selected from the group consisting of silicone acrylic hybrid polymers, silicone-based polymers, silicone gel adhesives, and polymers based on natural or synthetic rubbers. In particular, the polymer II may be a polymer or a mixture of polymers selected from the group consisting of silicone-based polymers and silicone gel adhesives.

In particular embodiments, the polymer II may be a silicone gel adhesive.

Also, the polymer II may be polymer or a mixture of polymers selected from silicone-based polymers, in particular from polymers based on polysiloxanes such as an amine-compatible polysiloxanes, or the polymer II may be a polymer or a mixture of polymers selected from natural or synthetic rubbers, in particular from styrenic triblock copolymers and/or polyisobutylenes, such as an SIS block copolymer and/or polyisobutylene.

Suitable polymers II according to the invention are commercially available e.g. under the brand names Soft skin adhesives (two-part silicone adhesive that cures upon mixing the two components). Alternative, suitable polymer II according to the invention are commercially available e.g. under the brand names BIO-PSA (pressure sensitive adhesives based on polysiloxanes) JSR-SIS (SIS block copolymer-based pressure-sensitive adhesives) and Oppanol™ (polyisobutylenes).

Additional polymers may also be added, e.g., to enhance adhesion of the skin contact layer.

According to some embodiments, the polymer II contained in the skin contact layer is different from the polymer I (i.e. the silicone-based polymer) contained in the active agent-containing layer. According to other embodiments, the polymer II contained in the skin contact layer is the same as the polymer I (i.e. the silicone-based polymer) contained in the active agent-containing layer.

According to certain embodiments, the area weight of the skin contact layer may range from 80 to 500 g/m². In a preferred embodiment, the skin contact layer has an area weight of from 100 to 350 g/m², from 150 to 320 g/m², or from 180 to 280 g/m². Such a relatively thick skin contact layer enables gentle removal of medical patches comprising the self-adhesive layer structure according to the invention, and thus is advantageous in reducing skin irritations. On the other hand, even if the elasticity of the skin contact layer and thus the ease of removability increases with its thickness, the total thickness of the self-adhesive layer structure should still allow for simple handling and wearing comfort. It was surprisingly found that the active release is not affected by such a thick (active free) skin contact layer, but on the contrary is even improved when compared to a medical patch without a skin contact layer.

### Silicone Acrylic Hybrid Polymer

Silicone acrylic hybrid polymers comprise a polymerized hybrid species that includes silicone-based sub-species and acrylate-based sub-species that have been polymerized together. The silicone acrylic hybrid polymer thus comprises a silicone phase and an acrylic phase. In certain embodiments, the silicone acrylic hybrid polymer is a silicone acrylic hybrid pressure-sensitive adhesive.

Silicone acrylic hybrid pressure-sensitive adhesives are usually supplied and used in solvents like n-heptane and ethyl acetate. The solids content of the pressure-sensitive adhesives is usually between 30% and 80%. The skilled person is aware that the solids content may be modified by adding a suitable amount of solvent.

The weight ratio of silicone to acrylate in the silicone acrylic hybrid pressure-sensitive adhesive may be from 5:95 to 95:5, or from 20:80 to 80:20, or from 40:60 to 60:40, or the ratio of silicone to acrylate may be about 50:50.

Suitable silicone acrylic hybrid pressure-sensitive adhesives which are commercially available include the PSA series 7-6100 and 7-6300 manufactured and supplied in n-heptane or ethyl acetate by Dow Corning (7-610X and 7-630X; X=1 n-heptane-based/X=2 ethyl acetate-based). For example, the 7-6102 silicone acrylic hybrid PSA having a silicone/acrylate ratio of 50/50 is characterized by a solution viscosity at 25° C. and about 50% solids content in ethyl acetate of 2,500 cP and a complex viscosity at 0.1 rad/s at 30° C. of 1.0e7 Poise. The 7-6302 silicone acrylic hybrid PSA having a silicone/acrylate ratio of 50/50 has a solution viscosity at 25° C. and about 50% solids content in ethyl acetate of 1,500 cP and a complex viscosity at 0.1 rad/s at 30° C. of 4.0e6 Poise.

Depending on the solvent in which the silicone acrylic hybrid pressure-sensitive adhesive is supplied, the arrangement of the silicone phase and the acrylic phase providing a silicone or acrylic continuous external phase and a corresponding discontinuous internal phase is different. If the silicone acrylic hybrid pressure-sensitive adhesive is provided in n-heptane, the composition contains a continuous, silicone external phase and a discontinuous, acrylic internal phase. If the silicone acrylic hybrid pressure-sensitive adhesive is provided in ethyl acetate, the composition contains a continuous, acrylic external phase and a discontinuous, silicone internal phase. After evaporating the solvent in which the silicone acrylic hybrid pressure-sensitive adhesive is provided, the phase arrangement of the resulting pressure-sensitive adhesive film or layer corresponds to the phase arrangement of the solvent-containing adhesive coating composition. For example, in the absence of any substance that may induce an inversion of the phase arrangement in a silicone acrylic hybrid pressure sensitive adhesive composition, a pressure-sensitive adhesive layer prepared from a silicone acrylic hybrid pressure-sensitive adhesive in n-heptane provides a continuous, silicone external phase and a discontinuous, acrylic internal phase, a pressure-sensitive adhesive layer prepared from a silicone acrylic hybrid pressure-sensitive adhesive in ethyl acetate provides a continuous, acrylic external phase and a discontinuous, silicone internal phase. The phase arrangement of the compositions can, for example, be determined in peel force tests with pressure-sensitive adhesive films or layers prepared from the silicone acrylic hybrid PSA compositions which are attached to a siliconized release liner. The pressure-sensitive adhesive film contains a continuous, silicone external phase if the siliconized release liner cannot or can only hardly be removed from the pressure-sensitive adhesive film (laminated to a backing film) due to the blocking of the two silicone surfaces. Blocking results from the adherence of two silicone layers which comprise a similar surface energy. The silicone adhesive shows a good spreading on the siliconized liner and therefore can create a good adhesion to the liner. If the siliconized release liner can easily be removed the pressure-sensitive adhesive film contains a continuous, acrylic external phase. The acrylic adhesive has no good spreading due to the different surface energies and thus has a low or almost no adhesion to the siliconized liner.

The silicone acrylic hybrid polymer may be a silicone acrylic hybrid pressure-sensitive adhesive obtainable from a silicone-containing pressure-sensitive adhesive composition comprising acrylate or methacrylate functionality. It is to be understood that the silicone-containing pressure-sensitive adhesive composition comprising acrylate or methacrylate functionality can include only acrylate functionality, only methacrylate functionality, or both acrylate functionality and methacrylate functionality.

The silicone acrylic hybrid pressure-sensitive adhesive may comprise the reaction product of (a) a silicone-containing pressure-sensitive adhesive composition comprising acrylate or methacrylate functionality, (b) an ethylenically unsaturated monomer, and (c) an initiator. That is, the silicone acrylic hybrid pressure-sensitive adhesive is the product of the chemical reaction between these reactants ((a), (b), and (c)). In particular, the silicone acrylic hybrid pressure-sensitive adhesive may include the reaction product of (a) a silicone-containing pressure-sensitive adhesive composition comprising acrylate or methacrylate functionality, (b) a (meth)acrylate monomer, and (c) an initiator (i.e., in the presence of the initiator). That is, the silicone acrylic hybrid pressure-sensitive adhesive may include the product of the chemical reaction between these reactants ((a), (b), and (c)).

The reaction product of (a) a silicone-containing pressure-sensitive adhesive composition comprising acrylate or methacrylate functionality, (b) an ethylenically unsaturated monomer, and (c) an initiator may contain a continuous, silicone external phase and a discontinuous, acrylic internal phase or the reaction product of (a), (b), and (c) may contain a continuous, acrylic external phase and a discontinuous, silicone internal phase.

The silicone acrylic hybrid polymer may comprise a reaction product of a silicone polymer, a silicone resin and an acrylic polymer, wherein the acrylic polymer is covalently self-crosslinked and covalently bound to the silicone polymer and/or the silicone resin.

The silicone acrylic hybrid polymer may comprise a reaction product of a silicone polymer, a silicone resin and an acrylic polymer, wherein the silicone resin contains triorganosiloxy units R₃SiO_{1/2} where R is an organic group, and tetrafunctional siloxy units SiO_{4/2} in a mole ratio of from 0.1 to 0.9 R₃SiO_{1/2} units for each SiO_{4/2}.

The acrylic polymer may comprise at least an alkoxysilyl functional monomer, polysiloxane-containing monomer, halosilyl functional monomer or alkoxy halosilyl functional monomer. In certain embodiments, the acrylic polymer is prepared from alkoxysilyl functional monomers selected from the group consisting of trialkoxylsilyl (meth)acrylates, dialkoxyalkylsilyl (meth)acrylates, and mixtures thereof, or comprises end-capped alkoxysilyl functional groups. The alkoxysilyl functional groups may preferably be selected from the group consisting of trimethoxylsilyl groups, dimethoxymethylsilyl groups, triethoxylsilyl, diethoxymethylsilyl groups and mixtures thereof.

The acrylic polymer may also be prepared from a mixture comprising polysiloxane-containing monomers, preferably from a mixture comprising polydimethylsiloxane mono (meth)acrylate.

The silicone acrylic hybrid polymer may be prepared by a) reacting silicone polymer with silicone resin to form a resultant product, b) reacting the resultant product of a) with an acrylic polymer containing reactive functionality, wherein the components are reacted in an organic solvent.

The silicone acrylic hybrid polymer may be prepared by a) reacting a silicone resin with an acrylic polymer containing reactive functionality to form a resultant product, b) reacting the resultant product of a) with silicone polymer, wherein the components are reacted in an organic solvent.

The silicone acrylic hybrid polymer may be prepared by a) reacting a silicone polymer with an acrylic polymer containing reactive functionality to form a resultant product, b) reacting the resultant product of a) with silicone resin, wherein the components are reacted in an organic solvent.

Further suitable acrylic polymers, silicone resins, and silicone polymers that can be used for chemically reacting together a silicone polymer, a silicone resin and an acrylic polymer to provide a silicone acrylic hybrid polymer in accordance with the previous paragraphs are detailed in WO 2010/124187.

### Silicone-Based Polymer

The silicone-based polymer is a non-curing polymer, which is typically applied by a hot-melt or a solvent-based process and preferably does not undergo further curing to solidify.

Silicone-based polymers are based on polysiloxanes. They may therefore also be referred to as polymers based on polysiloxanes. Silicone-based polymers are generally obtainable by polycondensation of silanol endblocked polydimethylsiloxane with a silicate resin. Amine-compatible silicone-based polymers can be obtained by reacting the silicone-based polymer with trimethylsilyl (e.g. hexamethyldisilazane) in order to reduce the silanol content of the polymer and thus provide enhanced stability in the presence of amines. As a result, the residual silanol functionality is at least partly, preferably mostly or fully capped with trimethylsiloxy groups.

Thus, in a preferred embodiment, the silicone-based polymer is an amine-compatible polysiloxane, and preferably is obtainable by polycondensation of a silanol endblocked polydimethylsiloxane with a silicate resin followed by at least partial trimethylsilylation of the residual silanol functionality.

In certain embodiments, the silicone-based polymer is pressure sensitive adhesive or a mixture of pressure sensitive adhesives, i.e. a pressure sensitive adhesive based on polysiloxanes or a mixture of pressure sensitive adhesives based on polysiloxanes

Pressure sensitive adhesives based on polysiloxanes provide for suitable tack and for quick bonding to various skin types, including wet skin, suitable adhesive and cohesive qualities, long lasting adhesion to the skin, a high degree of flexibility, a permeability to moisture, and compatibility to many actives and film-substrates. Such pressure sensitive adhesives are based on a resin-in-polymer concept wherein, by condensation reaction of silanol endblocked polydimethylsiloxane with a silica resin (also referred to as silicate resin), a pressure sensitive adhesive based on polysiloxane is prepared. For amine stability, the residual silanol functionality is additionally capped with trimethylsiloxy groups. The silanol endblocked polydimethylsiloxane content contributes to the viscous component of the visco-elastic behavior, and impacts the wetting and the spreadability properties of the adhesive. The resin acts as a tackifying and reinforcing agent, and participates in the elastic component. The correct balance between silanol endblocked polydimethylsiloxane and resin provides for the correct adhesive properties.

As indicated before, the tackiness of the silicone-based polymer may be modified by the resin-to-polymer ratio, i.e. the ratio of the silanol endblocked polydimethylsiloxane to the silicate resin, which is preferably in the range of from 50:50 to 70:30, or from 55:45 to 65:35. The tackiness will be increased with increasing amounts of the polydimethylsiloxane relative to the resin. High tack silicone-based polymers preferably have a resin-to-polymer ratio of 55:45, medium tack silicone-based polymers preferably have a resin-to-polymer ratio of 60:40, and low tack silicone-based polymers preferably have a resin-to-polymer ratio of 65:35.

According to certain embodiments, the pressure sensitive adhesive is obtainable by polycondensation of a silanol endblocked polydimethylsiloxane with a silicate resin, preferably with a resin-to-polymer ratio of from 50:50 to 70:30 or of 55:45, 60:40 or 65:35. Thus, in a preferred embodiment, the silicone-based polymer is a mixture of pressure sensitive adhesives obtainable by polycondensation of a silanol endblocked polydimethylsiloxane with a silicate resin with a resin-to-polymer ratio of 55:45 or of 60:40.

Further, according to certain embodiments, the silicone-based polymer is a mixture of pressure sensitive adhesives with
a solution viscosity at 25° C. and about 60% solids content in heptane of 450 mPa s and/or a complex viscosity at 0.01 rad/s at 30° C. of 1×10⁸ Poise, and
a solution viscosity at 25° C. and about 60% solids content in heptane of 500 mPa s and/or a complex viscosity at 0.01 rad/s at 30° C. of 5×10⁶ Poise.

The pressure sensitive adhesives based on polysiloxanes are supplied and used in solvents like n-heptane, ethyl acetate or other volatile silicone fluids. The solids content of pressure sensitive adhesives based on polysiloxanes in solvents is usually between 60 and 85%, between 70 and 80% or between 60 and 75%. The skilled person is aware that the solids content may be modified by adding a suitable amount of solvent.

High tack silicone-based polymers preferably have a complex viscosity at 0.01 rad/s and 30° C. of about 5×10⁶ Poise, medium tack silicone-based polymers preferably have a complex viscosity at 0.01 rad/s and 30° C. of about 5×10⁷ Poise, and low tack silicone-based polymers preferably have a complex viscosity at 0.01 rad/s and 30° C. of about 5×10⁸ Poise. High tack amine-compatible silicone-based polymers preferably have a complex viscosity at 0.01 rad/s and 30° C. of about 5×10⁶ Poise, medium tack amine-compatible silicone-based polymers preferably have a complex viscosity at 0.01 rad/s and 30° C. of about 5×10⁸ Poise, and low tack amine-compatible silicone-based polymers preferably have a complex viscosity at 0.01 rad/s and 30° C. of about 5×10⁹ Poise. Preferred pressure sensitive adhesives based on polysiloxanes in accordance with the invention are characterized by a solution viscosity at 25° C. and 60% solids content in n-heptane of more than about 150 mPa s, or from about 200 mPa s to about 700 mPa s, preferably as measured using a Brookfield RVT viscometer equipped with a spindle number 5 at 50 rpm. Theses may also be characterized by a complex viscosity at 0.01 rad/s at 30° C. of less than about 1×10⁹ Poise or from about 1×10⁵ to about 9×10⁸ Poise.

Suitable silicone-based polymers are commercially available under the brand names BIO-PSAs. Examples of silicone-based PSA compositions which are commercially available include the standard Liveo™ BIO-PSA series (7-4400,7-4500 and 7-4600 series) and the amine compatible (endcapped) Liveo™ BIO-PSA series (7-4100, 7-4200 and 7-4300 series) manufactured and typically supplied in n-heptane or ethyl acetate. For example, BIO-PSA 7-4201 is characterized by a solution viscosity at 25° C. and about 60% solids content in heptane of 450 mPa s and a complex viscosity at 0.01 rad/s at 30° C. of 1×10⁸ Poise. BIO-PSA 7-4301 has a solution viscosity at 25° C. and about 60% solids content in heptane of 500 mPa s and a complex viscosity at 0.01 rad/s at 30° C. of 5×10⁶ Poise.

Pressure sensitive adhesives based on polysiloxanes may be obtained according to the following scheme:

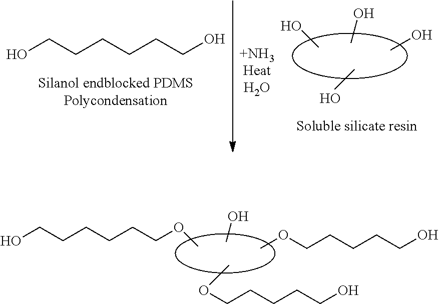

Such pressure sensitive adhesives based on polysiloxanes are available under the tradenames Liveo™ BIO-PSA 7-4401, BIO-PSA-7-4501, or BIO-PSA 7-4601, which are provided in the solvent n-heptane (indicated by the code “01”), or under the tradenames Liveo™ BIO-PSA 7-4402, BIO-PSA 7-4502, and BIO 7-4602, which are provided in the solvent ethyl acetate (indicated by the code “02”). Typical solids contents in the solvent are in the range of from 60 to 75%. The code “44” indicates a resin-to-polymer ratio of 65:35 resulting in a low tackiness, the code “45” indicates a resin-to-polymer ratio of 60:40 resulting in medium tackiness, the code “46” indicates a resin-to-polymer ratio of 55:45 resulting in high tackiness.

Amine-compatible pressure sensitive adhesives based on polysiloxanes may be obtained according to the following scheme:

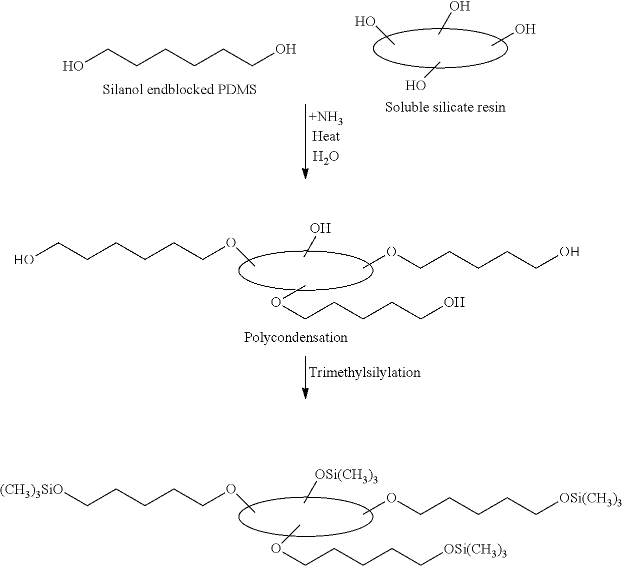

Such amine-compatible pressure sensitive adhesives based on polysiloxanes are available under the tradenames Liveo™ BIO-PSA 7-4101, BIO-PSA-7-4201, or BIO-PSA 7-4301, which are provided in the solvent n-heptane (indicated by the code “01”), or under the tradenames Liveo™ BIO-PSA 7-4102, BIO-PSA 7-4202, and BIO 7-4302, which are provided in the solvent ethyl acetate (indicated by the code “02”). Typical solids contents in the solvent are in the range of from 60 to 75%. The code “41” indicates a resin-to-polymer ratio of 65:35 resulting in a low tackiness, the code “42” indicates a resin-to-polymer ratio of 60:40 resulting in medium tackiness, the code “43” indicates a resin-to-polymer ratio of 55:45 resulting in high tackiness.

### Silicone Gel Adhesive

The silicone gel adhesive is an elastic, jelly-like material formed by lightly crosslinking silicone polymers. Thus, in contrast to the silicone-based polymers as used herein, the silicone gel adhesive is based on a curable gel producing composition. The silicone gel adhesive provides for the adhesiveness of the medical patch to the skin, while at the same time reducing the problem of skin irritation. Furthermore, the drug delivery of the medical patch is not negatively affected, surprisingly the skin permeation behavior is even improved.

Silicone gel adhesives are also referred to as silicone gels and, e.g., described in WO 2011/022199 A2.

The silicone gel adhesive is generally formed from linear or branched silicones having reactive groups thereon. Such reactive groups undergo a crosslinking reaction during curing. Examples of crosslinking reactions include the hydrosilylation reaction in which a silicone having an Si—H reactive group reacts with a silicone having an aliphatic unsaturated reactive group in the presence of a hydrosilylation catalyst. These materials are described, for example in U.S. Pat. Nos. 5,656,279, 5,891,076, EP 0 322 118 and U.S. Pat. No. 4,991,574 which are incorporated herein by reference. An alternative reaction is the condensation cure in which an alkoxy and/or hydroxy containing siloxanes are cured with a catalyst as described in U.S. Pat. No. 4,831,070 which is hereby incorporated by reference.

Typically, the silicone gel adhesive is obtainable by reacting a gel producing composition comprising (i) at least one alkenyl-substituted polydiorganosiloxane, (ii) at least one organosiloxane, which contains silicone-bonded hydrogen atoms, and (iii) at least one catalyst for the reaction of the SiH groups with the Si-alkenyl groups. These compositions cure at normal ambient temperatures, but curing can be expedited by heating to elevated temperatures, e.g., from 40 to 140° C., or by applying UV light.

Suitable alkenyl groups contain from 2 carbon to about 6 carbon atoms and are exemplified by, but not limited to, vinyl, allyl, and hexenyl. The alkenyl groups in this component may be located at terminal, pendant (non-terminal), or both terminal and pendant positions. The remaining silicone-bonded organic groups in the alkenyl-substituted polydiorganosiloxane are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms and are exemplified by, but not limited to, alkyl such as methyl, ethyl, propyl, and butyl; aryl such as phenyl; and halogenated alkyl such as 3,3,3-trifluoropropyl. Typically, at least 50 percent of the organic groups in the alkenyl-substituted polydiorganosiloxane are methyl. The structure of the alkenyl-substituted polydiorganosiloxane is typically linear, however, it may contain some branching due to the presence of trifunctional siloxane units. The viscosity of the alkenyl-substituted polydiorganosiloxane can be any desired. For example, it can be >0 mm²/s to 100,000 mm²/s, alternatively 50 mm²/s to 80,000 mm²/s, alternatively 300 mm²/s-3,000 mm²/s.

Methods for preparing the alkenyl-substituted polydiorganosiloxanes (i) of the present invention, such as condensation of the corresponding halosilanes or equilibration of cyclic polydiorganosiloxanes, are well known in the art.

The alkenyl-substituted polydiorganosiloxanes can be used in the gel producing composition in an amount of 10 wt. %-90 wt. % based on the weight of the composition, alternatively 40 wt. %-90 wt. %, alternatively 50 wt. %-80 wt. %. The amount of alkenyl groups present in the alkenyl-substituted polydiorganosiloxane is typically in the range of 0.05 wt. %-1 wt. %, alternatively 0.05 wt. % to 1 wt. % based on the weight of the alkenyl-substituted polydiorganosiloxane.

The organosiloxane containing silicone-bonded hydrogen atoms (ii) are also known in the art as described, for example in U.S. Pat. No. 3,983,298. The hydrogen atoms in this component may be located at terminal, pendant (non-terminal), or both terminal and pendant positions. The remaining silicone-bonded organic groups in this component are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms, and are exemplified by, but not limited to, alkyl such as methyl, ethyl, propyl, and butyl; aryl such as phenyl; and halogenated alkyl such as 3,3,3-trifluoropropyl. In one embodiment of the invention, at least 50 percent of the organic groups in the organosiloxane containing silicone-bonded hydrogen atoms are methyl. The structure of the organosiloxane containing silicone-bonded hydrogen atoms is typically linear however; it may contain some branching due to the presence of trifunctional siloxane units. The viscosity of the organosiloxane containing silicone-bonded hydrogen atoms can be any desired. For example, it can be >0 mm²/s to 100,000 mm²/s, alternatively, 5 mm²/s to 500 mm²/s.

Methods of preparing the organosiloxane containing silicone-bonded hydrogen atoms of the present invention by co-hydrolysis of the appropriate chlorosilanes are known in the art; U.S. Pat. No. 2,877,255 to Clark; Japanese Laid Open Patent Application (KOKAI) SHO 62(1987)-39660 to Mogi et al.; and U.S. Pat. Nos. 5,446,185 and 5,493,040 to Cobb et al., which are all hereby incorporated by reference.

The organosiloxanes containing silicone-bonded hydrogen atoms can be used in the gel producing composition in an amount of 1 wt. %-30 wt. % based on the weight of the composition, alternatively 5 wt. %-20 wt. %, and alternatively 5 wt. %-15 wt. %. In one embodiment, the amount of hydrogen group present in the organosiloxane containing silicone-bonded hydrogen atoms is between 0.05 wt. %-1.44 wt. % based on the weight of the organosiloxane containing silicone-bonded hydrogen atoms.

In the gel producing compositions, (i) and (ii) are preferably present such that the ratio of (H as SiH):(Alkenyl as Si-Alkenyl) is generally in the range of 0.1:1 to 10:1.

The hydrosilylation catalyst (iii) promotes the addition reaction of the alkenyl-substituted polydiorganosiloxane with the organosiloxane containing silicone-bonded hydrogen. The hydrosilylation catalyst can be any of the well-known hydrosilylation catalysts comprising a platinum group metal, a compound containing a platinum group metal, or a microencapsulated platinum group metal or compound containing same. These platinum group metals include platinum, rhodium, ruthenium, palladium, osmium and iridium. Platinum and platinum compounds are preferred catalysts based on their high activity level in hydrosilylation reactions. One class of platinum catalysts is the complexes of chloroplatinic acid with certain vinyl-containing organosiloxane compounds disclosed by Willig in U.S. Pat. No. 3,419,593, which is hereby incorporated by reference. A specific catalyst of this type is the reaction product of chloroplatinic acid and 1,3-diethenyl-1,1,3,3-tetramethyldisiloxane.

The hydrosilylation catalyst is present in an amount sufficient to cure the composition of the present invention. Typically, the concentration of the catalyst is sufficient to provide from 0.1 ppm to 500 ppm (part per million), alternatively from 1 ppm to 100 ppm, alternatively from 1 ppm to 50 ppm of a platinum group metal, based on the weight of (i) and (ii).

In view of the above, in a preferred embodiment of the invention, the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst, wherein preferably (i) and (ii) are present such that the ratio of (H as SiH):(Alkenyl as Si-Alkenyl) is generally in the range of 0.1:1 to 10:1.

An optional ingredient is a hydroxy substituted silicone resin as described in US. Patent Application No. 2007-0202245, herein incorporated by reference. The resin is typically comprised of groups having the formula R³₃SiO_{1/2} (“M” groups) and groups having the formula SiO_{4/2} (“Q” groups) where R³ is a alkyl group having 1 carbon to 6 carbon atoms or alkylene group having 1 carbon to 6 carbon atoms, typically methyl or vinyl. If an alkenyl group is present in the resin, typically the mol-% of R groups present as alkenyl groups is <10 mol %, alternatively 5 mol-%. The number ratio of M groups to Q groups is typically in the range of 0.6:1 to 4:1, alternatively 0.6:1 to 1.0:1. The silicone resin typically contains 0.1 wt % to 5 wt %, alternatively 1.0 wt % to 5 wt % silicone-bonded hydroxy groups.

The resin can be used in the gel producing composition in an amount of 2 wt. % to 45 wt. %, based on the weight of the gel producing composition and resin; alternatively 5 wt. % to 40 wt. %, alternatively 10 wt. % to 35 wt. %.

Thus, in a preferred embodiment, the silicone gel adhesive is a silicate resin-reinforced silicone gel adhesive that contains from about 2 to about 45% by weight of at least one hydroxyl substituted silicate resin.

According to particular embodiments, the silicone gel adhesive is a 2-component silicone adhesive system that cures upon mixing the two components. An example of such two-part silicone adhesive which is commercially available includes the Liveo™ Soft Skin Adhesives (e.g. MG 7-9700, MG 7-9800, MG 7-9850 and MG 7-9900) provided as a kit including components A and B. It is a platinum-catalyzed, soft, fillerless elastomeric silicone adhesive for adhering medical devices to the skin with medium adhesion force and gentle removal. The two components A and B are preferably mixed in a ratio of 1:1.

The silicone gel adhesive layer can be made by processes known in the art. For example, the gel may be pre-formed (e.g. as a sheet) by molding, calendaring, extruding, spraying, brushing, applying by hand, casting or coating on a substrate such as a liner. Or the silicone gel layer can be made by applying the gel producing composition to a substrate by spraying, coating, bar coating, etc. Once applied to the substrate the gel producing composition is cured to produce the silicone gel adhesive on the substrate.

### Polymer Based on Natural or Synthetic Rubbers

Polymers based on natural or synthetic rubbers include hydrocarbon polymers such as (natural and synthetic) polyisoprene, polybutylene and polyisobutylene, styrene/butadiene polymers, styrene-isoprene-styrene block copolymers, butyl rubber, halogen-containing polymers such as polyacrylic-nitrile, polytetrafluoroethylene, polyvinylchloride, polyvinylidene chloride, and polychlorodiene, other copolymers thereof. The polymers may in particular be used in combination with a tackifier as defined below.

According to certain embodiments, the polymer may be a styrenic triblock copolymer selected from the group consisting of styrene-ethylene-styrene (SES) block copolymers, styrene-butadiene-styrene (SBS) block copolymers, styrene-isoprene-styrene (SIS) block copolymers, styrene-ethylene/butylene-styrene (S-EB-S) block copolymers, styrene-ethylene/butylene/propylene-styrene (s-EBS-S) block copolymers, styrene-isoprene/butadiene-styrene (S—IB—S) block copolymers, and mixtures thereof.

In certain embodiments, the polymer may be at least one SIS block copolymer. The at least one SIS block copolymer may consist of three blocks of polystyrene, polyisoprene and polystyrene and in particular has a molecular weight of from about 100,000 to 200,000. In particular embodiments, the SIS block copolymer may comprise blocks of polystyrene and of polyisoprene in a ratio of from about 10:90(%) to about 30:70(%), or in a ratio of about 15:85(%) or about 22:78(%).

In other embodiments, the polymer is at least one polyisobutylene and may be a combination of two different types of polyisobutylenes, in particular a combination of low-molecular weight polyisobutylene and high-molecular weight polyisobutylene. In particular embodiments, the ratio of the low-molecular weight polyisobutylene to the high-molecular weight polyisobutylene is in the range of from 75:25 to 90:10.

Suitable SIS block copolymers according to the invention are commercially available e.g. under the brand names JSR-SIS. Specific SIS block copolymer-based pressure-sensitive adhesives are available under the tradenames JSR-SIS5229 and JSR-SIS5002.

Suitable polyisobutylenes according to the invention are commercially available e.g. under the tradename Oppanol®. Combinations of high-molecular weight polyisobutylenes (B100, B80) and low-molecular weight polyisobutylenes (B10, B11, B12, B13) may be used. Suitable ratios of low-molecular weight polyisobutylene to high-molecular weight polyisobutylene are in the range of from 100:1 to 1:100, from 95:5 to 40:60, or from 90:10 to 75:25. A particular example for a polyisobutylene combination is B10/B100 in a ratio of 85/15, or B12/B100 in a ratio of 80/20. Oppanol® B100 has a viscosity average molecular weight My of 1,110,000, and a weight average molecular weight Mw of 1,550,000, and an average molecular weight distribution Mw/Mn of 2.9. Oppanol® B10 has a viscosity average molecular weight My of 40,000, and a weight average molecular weight Mw of 53,000, and an average molecular weight distribution Mw/Mn of 3.2. Oppanol® B12 has a viscosity average molecular weight My of 55,000, and a weight average molecular weight Mw of 70,000, and an average molecular weight distribution Mw/Mn of 3.2. A suitable polyisobutylene adhesive is also commercially available e.g. under the brand name Duro-Tak™ 87-6908.

### Further Additives

The medical patch according to the invention, and in particular the active agent-containing layer may further comprise at least one additive or excipient. Said additives or excipients are preferably selected from the group consisting of crystallization inhibitors, solubilizers, fillers, substances for skincare, pH regulators, preservatives, tackifiers, softeners, stabilizers, and permeation enhancers, in particular from crystallization inhibitors, substances for skincare, tackifiers, softeners, stabilizers, and permeation enhancers. Such additives may be present in the active agent-containing layer in an amount of from 0.001 to 15% by weight, e.g. from 1 to 10% by weight or from 0.01 to 5% by weight, based on the total weight of the active agent-containing layer.

It should be noted that in pharmaceutical formulations, the formulation components are categorized according to their physicochemical and physiological properties, and in accordance with their function. This means in particular that a substance or a compound falling into one category is not excluded from falling into another category of formulation component. E.g. a certain polymer can be a crystallization inhibitor but also a tackifier. Some substances may e.g. be a typical softener but at the same time act as a permeation enhancer. The skilled person is able to determine based on his general knowledge in which category or categories of formulation component a certain substance or compound belongs to. In the following, details on the excipients and additives are provided which are, however, not to be understood as being exclusive. Other substances not explicitly listed in the present description may be as well used in accordance with the present invention, and substances and/or compounds explicitly listed for one category of formulation component are not excluded from being used as another formulation component in the sense of the present invention.

In certain embodiments, the medical patch, in particular the active agent-containing layer may further comprise a crystallization inhibitor. Suitable examples of crystallization inhibitors include polyvinylpyrrolidone, vinyl acetate/vinylpyrrolidone copolymer and cellulose derivatives. The crystallization inhibitor is preferably polyvinylpyrrolidone, more preferably soluble polyvinylpyrrolidone. The crystallization inhibitor may increase the solubility of capsaicin or inhibit the crystallization of capsaicin.

In certain embodiments, the medical patch, in particular the active agent-containing layer further comprise a stabilizer, wherein the stabilizer is preferably selected from tocopherol and ester derivatives thereof and ascorbic acid and ester derivatives thereof. Preferred stabilizers include sodium metabisulfite, ascorbyl esters of fatty acids such as ascorbyl palmitate, ascorbic acid, butylated hydroxytoluene, tocopherol, tocopheryl acetate and tocopheryl linoleate.

In certain embodiments, the medical patch, in particular the active agent-containing layer further comprise a softener/plasticizer. Exemplary softeners/plasticizers include linear or branched, saturated or unsaturated alcohols having 6 to 20 carbon atoms, triglycerides and polyethylene glycols.

In certain embodiments, the medical patch, in particular the active agent-containing layer further comprise a solubilizer. The solubilizer preferably improves the solubility of capsaicin in the active agent-containing layer. Preferred solubilizers include, e.g., glycerol-, polyglycerol-, propylene glycol- and polyoxyethylene-esters of medium chain and/or long chain fatty acids, such as glyceryl monolinoleate, medium chain glycerides and medium chain triglycerides, non-ionic solubilisers made by reacting castor oil with ethylene oxide, and any mixtures thereof which may further contain fatty acids or fatty alcohols, cellulose and methylcellulose and derivatives thereof such as hydroxypropylcellulose and hypromellose acetate succinate, various cyclodextrins and derivatives thereof, non-ionic tri-block copolymers having a central hydrophobic chain of polyoxypropylene flanked by two hydrophilic chains of polyoxyethylene known as poloxamers, water-soluble derivatives of vitamin E, pharmaceutical graded or agglomerated spherical isomalt, a polyethylene glycol, polyvinyl acetate and polyvinylcaprolactame-based graft copolymer, also abbreviated as PVAc-PVCap-PEG and known as Soluplus®, purified grades of naturally derived castor oil, of polyethylene glycol 400, of polyoxyethylene sorbitan monooleate (such as polysorbate 80) or of propylene glycols, diethylene glycol monoethyl ether, glucono-delta-lactone, maize and potato starch, as well as any of the below mentioned soluble polyvinylpyrrolidones, but also insoluble/cross-linked polyvinylpyrrolidones such as crospovidones. However, also the permeation enhancers mentioned below can act as solubilizers. Furthermore, also crystallization inhibitors may act as solubilizers.

In certain embodiments, the medical patch, in particular the active agent-containing layer may further comprise a pH regulator. Suitable pH regulators include mild acids and bases including amine derivatives, inorganic alkali derivatives, and polymers with basic or acidic functionality.

In certain embodiments, the medical patch, in particular the active agent-containing layer further comprises a preservative. Suitable preservatives include parabens, formaldehyde releasers, isothiazolinones, phenoxyethanol, and organic acids such as benzoic acid, sorbic acid, levulinic acid and anisic acid.

In certain embodiments, the medical patch, in particular the active agent-containing layer may further comprise a substance for skincare. Such substances may be used to avoid or reduce skin irritation as detectable by the dermal response score. Suitable substances for skincare include sterol compounds such as cholesterol, dexpanthenol, alpha-bisabolol, and antihistamines.

Fillers such as silica gels, titanium dioxide and zinc oxide may be used in conjunction with the silicone-based polymer in order to influence certain physical parameters, such as cohesion and bond strength, in the desired way.

In case the active agent-containing layer is required to have self-adhesive properties and one or more polymers is/are selected which does/do not provide sufficient self-adhesive properties, a tackifier is added. The tackifier may be selected from polyvinylpyrrolidone (which, due to its ability to absorb water, is able to maintain the adhesive properties of the matrix layer and thus can be regarded as a tackifier in a broad sense), triglycerides, polyethylene glycols, dipropylene glycol, resins, resin esters, terpenes, and derivatives thereof, ethylene vinyl acetate adhesives, dimethylpolysiloxanes and polybutenes, preferably polyvinylpyrrolidone and more preferably soluble polyvinylpyrrolidone.

In certain embodiments, the medical patch may further comprise a permeation enhancer. Permeation enhancers are substances, which influence the barrier properties of the stratum corneum in the sense of increasing the capsaicin permeability. Some examples of permeation enhancers are polyhydric alcohols such as dipropylene glycol, propylene glycol, and polyethylene glycol; oils such as olive oil, squalene, and lanolin; fatty ethers such as cetyl ether and oleyl ether, fatty acid esters such as isopropyl myristate; urea and urea derivatives such as allantoin, polar solvents such as dimethyldecylphosphoxide, methylcetylsulfoxide, dimethylaurylamine, dodecyl pyrrolidone, isosorbitol, dimethylacetonide, dimethylsulfoxide, decylmethylsulfoxide, and dimethylformamide, salicylic acid, amino acids, benzyl nicotinate, and higher molecular weight aliphatic surfactants such as lauryl sulfate salts. Other agents include oleic and linoleic acids, ascorbic acid, panthenol, butylated hydroxytoluene, tocopherol, tocopheryl acetate, tocopheryl linoleate, propyl oleate, and isopropyl palmitate. If the active agent-containing layer further comprises a permeation enhancer, the permeation enhancer is preferably selected from diethylene glycol monoethyl ether (Transcutol®), diisopropyl adipate, isopropyl myristate, isopropyl palmitate, lauryl lactate, and dimethylpropylene urea. It has been found that the medical patch according to the invention provides sufficient permeability of capsaicin even if no permeation enhancer is present. Therefore, in certain embodiments of the invention, the active agent-containing layer does not comprise a permeation enhancer.

### Release Characteristics

The medical patch according to the present invention is designed for topically administering capsaicin to create a local effect at a point of action, or for transdermally administering capsaicin to the systemic circulation, for a predefined period of time. While topically administering capsaicin relies on diffusion into the skin itself and does not necessitate transdermal release into the systemic circulation, in vitro skin permeation experiments still provide a good indication as to whether there is an effective topical release.

According to certain embodiments, the medical patch provides a cumulative permeated amount of capsaicin as measured in a Franz diffusion cell with dermatomed human skin of from 0.1 to 1.0 μg/cm² or of about 0.3 μg/cm² over a time period of about 60 minutes, and/or a skin permeation rate of capsaicin as measured in a Franz diffusion cell with dermatomed human skin of 0.06 μg/(cm² h) to 0.3 μg/(cm² h), or of 0.2 μg/(cm² h) to 1.2 μg/(cm² h), or of about 0.6 μg/(cm² h) after 60 minutes.

In a specific embodiment of the invention, the medical patch according to the invention as described above provides a skin permeation rate of capsaicin as measured in a Franz diffusion cell with dermatomed human skin of
0 μg/(cm² h) to 0.9 μg/(cm² h) in the first 60 minutes,
0.6 μg/(cm² h) to 1.2 μg/(cm² h) from minute 60 to minute 90,
0.8 μg/(cm² h) to 1.4 μg/(cm² h) from minute 90 to minute 120,
0.9 μg/(cm² h) to 1.6 μg/(cm² h) from minute 120 to minute 240.

In particular embodiments, the medical patch provides therapeutically effective skin permeation of capsaicin, within less than 90 minutes, or less than 60 minutes after application of the medical patch to the skin.

### Method of Treatment/Medical Use

The medical patch according to the present invention is suitable for use in a method of treatment, and in particular in a method of treating a human patient.

In certain embodiments, the medical patch according to the invention is for use in a method of treatment, wherein the medical patch is preferably applied to the skin of the patient for less than 90 minutes, less than 60 minutes, or less than 30 minutes. In certain embodiments, the medical patch according to the invention is for use in a method of treatment with a dosing interval of at least or about 1.5 months, at least or about 2 months, or at least or about 3 months. Thus, it is preferred for a medical patch to be only applied after a break of at least 90 days after removal a preceding medical patch.

In a preferred embodiment, the medical patch according to the invention is for use in a method of treating neuropathic pain, in particular chronic neuropathic pain, preferably including postherpetic neuralgia, post-surgical neuralgia such as, e.g., post-herniotomy pain, post-thoracotomy pain or post-mastectomy pain, post-traumatic neuropathy, polyneuropathy such as, e.g., painful diabetic neuropathy, chemotherapy-induced neuropathy, neuropathy caused by tumors, HIV-associated neuropathy, alcohol-related neuropathy, small-fiber neuropathy or complex regional pain syndrome, radiculopathy, or compression syndromes such as carpal tunnel syndrome.

The medical patch according to the invention is further preferably for use in a method of treating peripheral neuropathic pain, neuropathic pain associated with postherpetic neuralgia or diabetic peripheral neuropathy (DPN) of the hands and feet, post-surgical neuropathic pain, joint pain, or cancer pain.

In certain embodiments, the medical patch according to the invention is for use in a method of treating neuropathic pain associated with postherpetic neuralgia, or neuropathic pain associated with diabetic peripheral neuropathy of the hands or feet in a human patient, in particular an adult. In certain embodiments, the medical patch according to the invention is for use in a method of treating post-surgical neuropathic pain in a human patient, in particular an adult.

In connection with the above, the medical patch according to the invention is preferably applied to at least one body surface on the patient, in particular selected from the back, the bottom, the legs, the feet, or the hands. The preferred application time of a medical patch according to the invention is less than or about 60 minutes on the back, bottom or legs, and less than or about 30 minutes on the feet or hands.

According to a specific aspect, the present invention is also related to a method of treating neuropathic pain, in particular chronic neuropathic pain, and preferably peripheral neuropathic pain, neuropathic pain associated with postherpetic neuralgia or diabetic peripheral neuropathy (DPN) of the hands or feet, post-surgical neuropathic pain, joint pain, or cancer pain including applying a medical patch as described herein to the skin of a patient.

### Process of Manufacture

The medical patch according to the present invention may be manufactured using a process comprising the steps of
1.1) coating an active agent-containing coating composition comprising
   (i) capsaicin, and
   (ii) at least one silicone-based polymer on a first foil;
1.2) drying the coated coating composition to form the active agent-containing layer;
1.3) laminating the active agent-containing layer with a backing layer.

The silicone-based polymer is preferably non-curing and therefore typically applied by a solvent-based process. Accordingly, the at least one silicone-based polymer is preferably provided in a solvent, wherein the solids content in the solvent is preferably from 40 to 75% by weight. The solvent is preferably selected from alcoholic solvents, in particular methanol, ethanol, isopropanol and mixtures thereof, and from non-alcoholic solvents, in particular ethyl acetate, hexane, heptane, petroleum ether, toluene, and mixtures thereof, and is more preferably selected from non-alcoholic solvents, and is most preferably ethyl acetate or n-heptane.

The capsaicin is preferably homogeneously dissolved or dispersed in the active agent-containing coating composition. According to certain embodiments, the capsaicin is provided in an amphiphilic solvent, such as, e.g., diethylene glycol monoethyl ether, 1,3-butanediol, dipropylene glycol or 2, 2-dimethyl-4-hydroxymethyl-1, 3-dioxolane, and the capsaicin preparation is dispersed in the active agent-containing coating composition in the form of small droplets (microreservoir system). The amphiphilic solvent must not mix or may only mix to a small extend with the solvent for the silicone-based polymer.

The coated active agent-containing coating composition is solidified by drying. Drying is preferably performed at a temperature of from 20 to 60° C., or from 30 to 40° C.

The process may further comprise the steps of
2.1) coating an active-free coating composition comprising
   (i) at least one alkenyl-substituted polydiorganosiloxane,
   (ii) at least one organosiloxane, which contains silicone-bonded hydrogen atoms, and
   (iii) at least one catalyst for the reaction of the SiH groups with the Si-alkenyl groups, on a second foil;
2.2) crosslinking the active-free coating composition at a temperature of from 50° C. to 150° C. or by applying UV light to form the skin contact layer;
2.3) laminating the skin contact layer with a release liner.

The active-free coating composition forms the silicone gel adhesive of the skin contact layer upon curing, i.e. crosslinking of the reactive groups of the silicone polymers. Crosslinking is preferably performed at a temperature of from 40° C. to 140° C.

The active agent-containing layer and the skin contact layer are preferably prepared separately as indicated above, and then laminated together by removing the foils and then laminating the open sides of the two layers together, so as to give an active agent-containing layer structure. Accordingly, the process may further comprise the steps of
3.1) removing the foils from the active agent-containing layer and the skin contact layer; and
3.2) laminating the open side of the active agent-containing layer onto the open side of the skin contact layer to obtain an active agent-containing layer structure.
   The preparation of the active agent-containing layer may be performed before or after the preparation of the skin contact layer, or the preparation of the two layers may be performed in parallel.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Example 1 and Reference Example

The skin permeation rate and utilization of capsaicin for the medical patch of Example 1 and the Reference Example were determined by in vitro experiments.

The medical patch according to Example 1 differs from the medical patch according to the Reference Example by an additional skin contact layer. The two medical patches were thus prepared equally, except that the steps of preparing and coating an active-free coating composition and laminating the resulting active-free layer with the previously prepared capsaicin-containing layer have not been carried out for the Reference Example.

### Capsaicin-Containing Coating Composition

For both Example 1 and the Reference Example, the formulation of the capsaicin-containing coating composition is summarized in Table 1.1 below. The solids %-values refer to the amounts (Amt) in % by weight.

TABLE 1.1

| Ingredient (Trade Name) | Solids [%] |
|---|---|
| Capsaicin | 8 |
| Polysiloxanes in n-heptane. Solids content of | 70 |
| 70% by weight (Liveo ™ BIO-PSA 7-4301 | |
| and BIO-PSA 7-4201) | |
| Transcutol (diethylene glycol monoethyl ether) | 19.2 |
| Ethyl cellulose | 0.8 |
| Silicone oil 360MED Fluid 12.500 cSt. | 2 |
| Total | 100 |
| Area Weight [g/m²] | 80 |
| Capsaicin content [μg/cm²] | 640 |

### Preparation of the Capsaicin-Containing Coating Composition

Transcutol was initially thickened with the ethyl cellulose under stirring (100-300 rpm).

A vessel was loaded with the polysiloxane mixture and the silicone oil and stirred (100-300 rpm) at least for 5 min before the ethyl cellulose/Transcutol solution was added. After further 10 min of stirring (100-300 rpm) capsaicin was added. The mixture was then stirred at approx. 250-300 rpm until a homogeneous mixture was obtained (at least 60 min).

### Coating of the Capsaicin-Containing Coating Composition

The resulting capsaicin-containing coating composition was coated on a fluoropolymer coated polyester film (Scotchpak™ 1022). The solvent was removed at room temperature for about 20-30 min.

The coating thickness was chosen such that removal of the solvent results in an area weight of the capsaicin-containing layer of about 80 g/m².

The resulting capsaicin-containing microreservoir layer was then laminated with a backing layer (polyester film, 19 μm).

### Active-Free Coating Composition

For Example 1, the formulation of the active-free coating composition is summarized in Table 1.2 below. The solids %-values refer to the amounts (Amt) in % by weight.

TABLE 1.2

| Ingredient (Trade Name) | Amt [g] | Solids [%] |
|---|---|---|
| Component A of Soft Skin Adhesive Kit | 25 | 50 |
| (Liveo ™ MG 7-9900 (A)) | | |
| Component B of Soft Skin Adhesive Kit | 25 | 50 |
| (Liveo ™ MG 7-9900 (B)) | | |
| Total | 50 | 100 |

| | |
|---|---|
| Area Weight [g/m²] | 230 |

### Preparation of the Active-Free Coating Composition

Both components were weighed separately, and then component A was added to the mixing vessel followed by component B. Then the mixture was mixed at approx. 100 rpm for approx. 10 min until a homogeneous mixture of Component A and Component B was obtained.

### Coating of the Active-Free Coating Composition

Within a time frame of approx. 30 min, the resulting active-free coating composition was coated on an adhesively equipped foil. The coating temperature was set to 120° C. The resulting active-free layer was heated at this temperature for approx. 40 min.

The coating thickness was chosen such that removal of the solvents resulted in a layer thickness of the active-free (skin contact) layer of approx. 230 g/m².

The resulting active-free (skin contact) layer was laminated with a release liner (FEP, fluorinated ethylene propylene, 125 μm).

### Lamination of the Capsaicin-Containing Layer and the Active-Free (Skin Contact) Layer

The active-free (skin contact) layer was then laminated with the capsaicin-containing layer. For this purpose, the adhesively equipped foils used for the coating and drying of the layers were removed and the resulting open sides of the active-containing layer and the active-free (skin contact) layer were laminated together resulting in a capsaicin-containing self-adhesive layer structure comprising the backing layer, the capsaicin-containing layer, and the active-free (skin contact) layer, wherein the capsaicin-containing layer is attached to the backing layer, and the active-free (skin contact) layer is attached to the capsaicin-containing layer, and wherein the structure is closed by a release liner, which is attached to the active-free (skin contact) layer.

### Preparation of the Medical Patch

The individual medical patches were punched out from the capsaicin-containing self-adhesive layer structure obtained as described above. Then, the medical patches were sealed into pouches of the primary packaging material.

### Measurement of the Skin Permeation

The permeated amount of medical patches prepared according to Example 1 and the Reference Example were determined by in vitro experiments in accordance with the OECD Guideline (adopted Apr. 13, 2004) carried out with a 10.0 ml Franz diffusion cell. Split thickness human skin from cosmetic surgeries (female abdomen, date of birth 1981) was used. A heat separated epidermis was used for all medical patches. Diecuts with an area of release of 1.171 cm² were punched from the medical patch. The capsaicin permeated amount in the receptor medium of the Franz cell (0.9% sodium chloride solution with 0.1% saline azide as antibacteriological agent) at a temperature of 32+1° C. was measured and the corresponding skin permeation rate calculated.

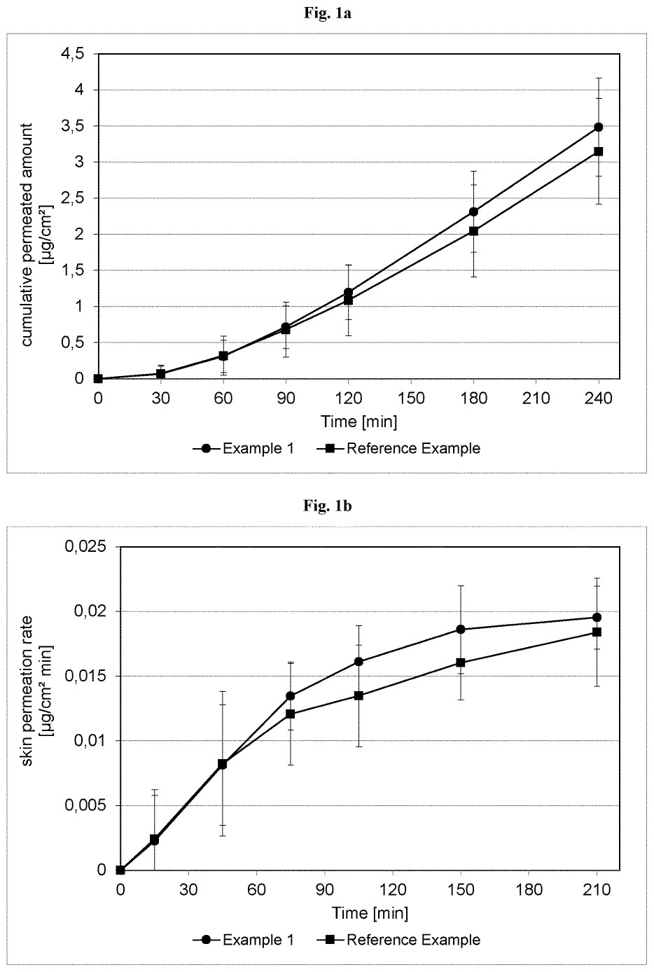

The results are shown in Table 1.3 and FIG. 1A.

TABLE 1.3
Cumulative permeated amount with SD* [μg/cm²]

| | | |
|---|---|---|
| Elapsed | Ex. 1 (n = 6) | Ref. Ex. (n = 6) |

| time [min] | Rate | SD | Rate | SD |
|---|---|---|---|---|
| 30 | 0.07 | 0.11 | 0.07 | 0.11 |
| 60 | 0.31 | 0.23 | 0.32 | 0.27 |
| 90 | 0.72 | 0.30 | 0.68 | 0.38 |
| 120 | 1.20 | 0.38 | 1.09 | 0.49 |
| 180 | 2.31 | 0.56 | 2.05 | 0.64 |
| 240 | 3.49 | 0.68 | 3.15 | 0.73 |

*Standard deviation was calculated based on the n-method.

The corresponding skin permeation rates (A flux rate) were calculated based on the respective permeated amount.

The results are shown in Table 1.4 and FIG. 1B.

TABLE 1.4
Skin permeation rate (Δ flux rate) with SD* [μg/cm² min]

| | | |
|---|---|---|
| Elapsed | Ex. 1 (n = 6) | Ref. Ex. (n = 6) |

| time [min] | Rate | SD | Rate | SD |
|---|---|---|---|---|
| 15 | 0.0023 | 0.0035 | 0.0024 | 0.0038 |
| 45 | 0.0081 | 0.0047 | 0.0082 | 0.0056 |
| 75 | 0.0135 | 0.0026 | 0.0121 | 0.0039 |
| 105 | 0.0161 | 0.0028 | 0.0135 | 0.0039 |
| 150 | 0.0186 | 0.0034 | 0.0160 | 0.0029 |
| 210 | 0.0195 | 0.0024 | 0.0184 | 0.0042 |

*Standard deviation was calculated based on the n-method.

The above results show that the skin permeation rate of the inventive capsaicin medical patch according to Example 1 surprisingly is almost identical to slightly higher/faster when compared to an identical patch just without the skin contact layer (in accordance with the Reference Example). As also outlined above, this means that the inventive patches could be based on already approved and marketed capsaicin patches, which are merely supplemented with a further skin contact layer, likely without changing the (approved) drug release behaviour of the commercial product, which is highly advantageous.

### The Invention Relates in Particular to the Following Further Items

1. Medical patch for the administration of capsaicin comprising an active agent-containing layer structure, said active agent-containing layer structure comprising:
   A) a backing layer;
   B) an active agent-containing layer comprising
      (i) capsaicin, and
      (ii) at least one silicone-based polymer;
   and
   C) a skin contact layer;
wherein the skin contact layer is an adhesive layer comprising a polymer II.
2. Medical patch according to item 1, wherein
the polymer II is a polymer or a mixture of polymers in which the capsaicin is substantially insoluble.
3. Medical patch according to item 1 or 2, wherein the polymer II is a pressure sensitive adhesive or a mixture of pressure sensitive adhesives.
4. Medical patch according to any one of items 1 to 3, wherein
the polymer II is a polymer or a mixture of polymers selected from the group consisting of silicone acrylic hybrid polymers, silicone-based polymers, silicone gel adhesives, and polymers based on natural or synthetic rubbers.
5. Medical patch according to any one of items 1 to 4, wherein
the polymer II is a polymer or a mixture of polymers selected from the group consisting of silicone-based polymers and silicone gel adhesives.
6. Medical patch according to any one of items 1 to 5, wherein
the polymer II is a silicone gel adhesive.
7. Medical patch according to item 6, wherein
the silicone gel adhesive is obtainable by reacting a gel producing composition comprising (i) at least one alkenyl-substituted polydiorganosiloxane, (ii) at least one organosiloxane, which contains silicone-bonded hydrogen atoms, and (iii) at least one catalyst for the reaction of the SiH groups with the Si-alkenyl groups.
8. Medical patch according to item 6 or 7, wherein
the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.
9. Medical patch according to any one of items 6 to 8, wherein
the silicone gel adhesive is a silicate resin-reinforced silicone gel adhesives that contain from about 2 to about 45% by weight of at least one hydroxyl substituted silicate resin.
10. Medical patch according to any one of items 1 to 9, wherein
the saturation concentration of the capsaicin in the skin contact layer is less than 0.1% by weight, less than 0.05% by weight, less than 0.02% by weight, or less than 0.01% by weight.
11. Medical patch according to any one of items 1 to 10, wherein
the area weight of the skin contact layer ranges from 100 to 350 g/m², from 150 to 320 g/m², or from 180 to 280 g/m².
12. Medical patch according to any one of items 1 to 11, wherein
the skin contact layer comprises the polymer II in an amount of at least 95% by weight, at least 99% by weight, or in an amount of about 100% by weight, based on the total weight of the skin contact layer.
13. Medical patch according to any one of items 1 to 12, wherein
the skin contact layer comprises the capsaicin in an amount of less than 0.1% by weight, or less than 0.01% by weight, based on the total weight of the skin contact layer.
14. Medical patch according to any one of items 1 to 13, wherein
the active agent-containing layer comprises the capsaicin in an amount of from 2 to 20% by weight, from 5 to 15% by weight, or from 5 to 10% by weight.
15. Medical patch according to item 14, wherein
the active agent-containing layer comprises the capsaicin in an amount of about 8% by weight.
16. Medical patch according to any one of items 1 to 15, wherein
the active agent-containing layer contains at least 0.30 mg/cm², at least 0.50 mg/cm², or at least 0.60 mg/cm² capsaicin.
17. Medical patch according to any one of items 1 to 16, wherein
the active agent-containing layer contains less than 1.0 mg/cm², less than 0.8 mg/cm², or less than 0.7 mg/cm² capsaicin.
18. Medical patch according to any one of items 1 to 17, wherein
the medical patch comprises the capsaicin in an amount of from 0.5 to 180 mg, from 1.2 to 90 mg, or 19 to 45 mg.
19. Medical patch to any one of items 1 to 18, wherein
the medical patch comprises the capsaicin in an amount of about 179 mg, of about 60 mg, of about 45 mg, of about 30 mg, of about 25 mg, of about 10 mg, or of about 1 mg.
20. Medical patch according to any one of items 1 to 19, wherein
the at least one silicone-based polymer is present in the active agent-containing layer in an amount of from 20 to 90% by weight, or 60 to 90% by weight, based on the total weight of the active agent-containing layer.
21. Medical patch according to any one of items 1 to 20, wherein
the silicone-based polymer is a pressure sensitive adhesive or a mixture of pressure sensitive adhesives.
22. Medical patch according to item 21, wherein
the pressure sensitive adhesive is obtainable by polycondensation of a silanol endblocked polydimethylsiloxane with a silicate resin, preferably with a resin-to-polymer ratio of from 50:50 to 70:30, or of 55:45, 60:40 or 65:35.
23. Medical patch according to any one of items 1 to 22, wherein
the silicone-based polymer is a mixture of pressure sensitive adhesives obtainable by polycondensation of a silanol endblocked polydimethylsiloxane with a silicate resin with a resin-to-polymer ratio of 55:45 or of 60:40.
24. Medical patch according to any one of items 1 to 23, wherein
the silicone-based polymer is a mixture of pressure sensitive adhesives with
a solution viscosity at 25° C. and about 60% solids content in heptane of 450 mPa s and/or a complex viscosity at 0.01 rad/s at 30° C. of 1×10⁸ Poise, and
a solution viscosity at 25° C. and about 60% solids content in heptane of 500 mPa s and/or a complex viscosity at 0.01 rad/s at 30° C. of 5×10⁶ Poise.
25. Medical patch according to any one of items 1 to 24, wherein
the silicone-based polymer is an amine-compatible polysiloxane, and preferably is obtainable by polycondensation of a silanol endblocked polydimethylsiloxane with a silicate resin followed by at least partial trimethylsilylation of the residual silanol functionality.
26. Medical patch according to any one of items 1 to 25, wherein
the active agent-containing layer comprises diethylene glycol monoethyl ether, and preferably comprises diethylene glycol monoethyl ether in an amount of from 10 to 25% by weight.
27. Medical patch according to any one of items 1 to 26, wherein
the active agent-containing layer comprises a viscosity-increasing additive selected from the group consisting of cellulose derivatives and high molecular weight polyacrylic acids, and any mixture thereof.
28. Medical patch according to any one of items 1 to 27, wherein
the active agent-containing layer comprises ethyl cellulose, and preferably comprises ethyl cellulose in an amount of from 0 to 2% by weight.
29. Medical patch according to any one of items 1 to 28, wherein
the active agent-containing layer comprises silicone oil, and preferably comprises silicone oil in an amount of from 0 to 5% by weight.
30. Medical patch according to any one of items 1 to 29, wherein
the area weight of the active agent-containing layer ranges from 30 to 200 g/m², or from 50 to 120 g/m 2
31. Medical patch according to any one of items 1 to 30, wherein
the backing layer is impermeable to capsaicin.
32. Medical patch according to any one of items 1 to 31, wherein
the backing layer consists of a polyester film, preferably with a thickness of 10-20 μm.
33. Medical patch according to any one of items 1 to 31, wherein
the backing layer consists of an ethylene-vinyl acetate copolymer.
34. Medical patch according to any one of items 1 to 33, wherein
the skin contact layer does not comprise capsaicin, and/or the medical patch further comprises a release liner.
35. Medical patch according to any one of items 1 to 34, wherein
the skin contact layer is directly attached to the active agent-containing layer.
36. Medical patch according to any one of items 1 to 34, wherein
the active agent-containing layer structure comprises a membrane which is located between the active agent-containing layer and the skin contact layer, wherein the membrane is preferably a rate controlling membrane.
37. Medical patch according to any one of items 1 to 36, wherein
the active agent-containing layer structure has a hexagonal shape, wherein the backing layer, the active agent-containing layer and the skin contact layer are coextensive and provide the hexagonal shape of the active agent-containing layer structure, and
the hexagonal shape comprises at least one hexagon, wherein
   all pairs of opposite sides of the hexagon are parallel, and
   the sides of the hexagon have a length of from 1.5 to 10 cm.
38. Medical patch according to item 37, wherein
the at least one hexagon is at least one convex hexagon.
39. Medical patch according to item 38, wherein
the hexagonal shape comprises one or two convex hexagons.
40. Medical patch according to any one of items 37 to 39, wherein
the hexagonal shape is a convex hexagon.
41. Medical patch according to any one of items 37 to 40, wherein
the hexagonal shape is a double-hexagon formed of two identical convex hexagons sharing two adjacent vertices and their common side.
42. Medical patch according to item 41, wherein
said common side is perforated for easy tear-off.
43. Medical patch according to any one of items 1 to 42, wherein
the medical patch provides
   a cumulative permeated amount of capsaicin as measured in a Franz diffusion cell with dermatomed human skin of from 0.1 to 1.0 μg/cm² or of about 0.3 μg/cm² over a time period of about 60 minutes, and/or
   a skin permeation rate of capsaicin as measured in a Franz diffusion cell with dermatomed human skin of 0.06 μg/(cm² h) to 0.3 μg/(cm² h), or of 0.2 μg/(cm² h) to 1.2 μg/(cm² h), or of about 0.6 μg/(cm² h) after 60 minutes.
44. Medical patch according to any one of items 1 to 43, wherein the medical patch is a topical medical patch.
45. Medical patch according to any one of items 1 to 43, wherein the medical patch is a transdermal therapeutic system.
46. Medical patch according to any one of items 1 to 45, wherein
the medical patch provides a skin permeation rate of capsaicin as measured in a Franz diffusion cell with dermatomed human skin of
   0 μg/(cm² h) to 0.9 μg/(cm² h) in the first 60 minutes,
   0.6 μg/(cm² h) to 1.2 μg/(cm² h) from minute 60 to minute 90,
   0.8 μg/(cm² h) to 1.4 μg/(cm² h) from minute 90 to minute 120,
   0.9 μg/(cm² h) to 1.6 μg/(cm² h) from minute 120 to minute 240.
47. Medical patch according to any one of items 1 to 46,
for use in a method of treating neuropathic pain, in particular chronic neuropathic pain, and preferably for use in a method of treating peripheral neuropathic pain, neuropathic pain associated with postherpetic neuralgia or diabetic peripheral neuropathy (DPN) of the hands and feet, post-surgical neuropathic pain, joint pain, or cancer pain.
48. Use of a medical patch according to any one of items 1 to 46,
for the manufacture of a medicament for treating neuropathic pain, in particular chronic neuropathic pain, and preferably for the manufacture of a medicament for treating peripheral neuropathic pain, neuropathic pain associated with postherpetic neuralgia or diabetic peripheral neuropathy (DPN) of the hands or feet, post-surgical neuropathic pain, joint pain, or cancer pain.
49. A method of treating neuropathic pain, in particular chronic neuropathic pain, and preferably peripheral neuropathic pain, neuropathic pain associated with postherpetic neuralgia or diabetic peripheral neuropathy (DPN) of the hands or feet, post-surgical neuropathic pain, joint pain, or cancer pain including applying a medical patch according to any one of items 1 to 46 to the skin of a patient.
50. Medical patch for the administration of capsaicin comprising an active agent-containing layer structure, said active agent-containing layer structure comprising:
   A) a backing layer;
   B) an active agent-containing layer comprising
      (i) capsaicin, and
      (ii) at least one silicone-based polymer;
   and
   C) a skin contact layer;
wherein the skin contact layer is an adhesive layer comprising a silicone gel adhesive.
51. Medical patch for the administration of capsaicin comprising an active agent-containing layer structure, said active agent-containing layer structure comprising:
   A) a backing layer;
   B) an active agent-containing layer comprising
      (i) capsaicin in an amount of from 5 to 10% by weight,
      (ii) at least one amine-compatible polysiloxane in an amount of from 60 to 90% by weight,
      (iii) diethylene glycol monoethyl ether in an amount of from 10 to 25% by weight,
      (iv) ethyl cellulose in an amount of from 0 to 2% by weight, and
      (v) silicone oil in amount of from 0 to 5% by weight;
   and
   C) a skin contact layer;
wherein the skin contact layer is an adhesive layer comprising a silicone gel adhesive, and wherein the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.
52. Medical patch for the administration of capsaicin comprising an active agent-containing layer structure, said active agent-containing layer structure comprising:
   A) a backing layer;
   B) an active agent-containing layer comprising
      (i) capsaicin in an amount of about 8% by weight,
      (ii) at least one amine-compatible polysiloxane in an amount of from 60 to 90% by weight,
      (iii) diethylene glycol monoethyl ether in an amount of from 10 to 25% by weight,
      (iv) ethyl cellulose in an amount of from 0 to 2% by weight, and
      (v) silicone oil in amount of from 0 to 5% by weight;
   and
   C) a skin contact layer;
wherein the skin contact layer is an adhesive layer comprising a silicone gel adhesive, and wherein the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.

1. Medical patch for the administration of capsaicin comprising an active agent-containing layer structure, said active agent-containing layer structure comprising:
A) a backing layer;
B) an active agent-containing layer comprising
(i) capsaicin, and
(ii) at least one silicone-based polymer;
and
C) a skin contact layer;
wherein the skin contact layer is an adhesive layer comprising a polymer II.

2. Medical patch according to claim 1, wherein
the polymer II is a polymer or a mixture of polymers selected from the group consisting of silicone acrylic hybrid polymers, silicone-based polymers, silicone gel adhesives, and polymers based on natural or synthetic rubbers, in particular from the group consisting of silicone-based polymers and silicone gel adhesives.

3. Medical patch according to claim 1 or 2, wherein
the polymer II is a silicone gel adhesive.

4. Medical patch according to claim 3, wherein
the silicone gel adhesive is obtainable by reacting a gel producing composition comprising (i) at least one alkenyl-substituted polydiorganosiloxane, (ii) at least one organosiloxane, which contains silicone-bonded hydrogen atoms, and (iii) at least one catalyst for the reaction of the SiH groups with the Si-alkenyl groups.

5. Medical patch according to claim 3 or 4, wherein
the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.

6. Medical patch according to any one of claims 3 to 5, wherein
the silicone gel adhesive is a silicate resin-reinforced silicone gel adhesive that contains from about 2 to about 45% by weight of at least one hydroxyl substituted silicate resin.

7. Medical patch according to any one of claims 1 to 6, wherein
the saturation concentration of the capsaicin in the skin contact layer is less than 0.1% by weight, less than 0.05% by weight, less than 0.02% by weight, or less than 0.01% by weight.

8. Medical patch according to any one of claims 1 to 7, wherein
the area weight of the skin contact layer ranges from 100 to 350 g/m², from 150 to 320 g/m², or from 180 to 280 g/m².

9. Medical patch according to any one of claims 1 to 8, wherein
the active agent-containing layer comprises the capsaicin in an amount of from 2 to 20% by weight, from 5 to 15% by weight, or from 5 to 10% by weight, or of about 8% by weight.

10. Medical patch according to any one of claims 1 to 9, wherein
the active agent-containing layer contains at least 0.30 mg/cm², at least 0.50 mg/cm², or at least 0.60 mg/cm² capsaicin, and/or
wherein the active agent-containing layer contains less than 1.0 mg/cm², less than 0.8 mg/cm², or less than 0.7 mg/cm² capsaicin.

11. Medical patch according to any one of claims 1 to 10, wherein
the medical patch comprises the capsaicin in an amount of from 0.5 to 180 mg, from 1.2 to 90 mg, or 19 to 45 mg.

12. Medical patch according to any one of claims 1 to 11, wherein
the area weight of the active agent-containing layer ranges from 30 to 200 g/m², or from 50 to 120 g/m 2.

13. Medical patch according to any one of claims 1 to 12, wherein
the skin contact layer is directly attached to the active agent-containing layer.

14. Medical patch according to any one of claims 1 to 12, wherein
the active agent-containing layer structure comprises a membrane which is located between the active agent-containing layer and the skin contact layer, wherein the membrane is preferably a rate controlling membrane.

15. Medical patch according to any one of claims 1 to 14, wherein
the medical patch provides
a cumulative permeated amount of capsaicin as measured in a Franz diffusion cell with dermatomed human skin of from 0.1 to 1.0 μg/cm² or of about 0.3 μg/cm² over a time period of about 60 minutes, and/or
a skin permeation rate of capsaicin as measured in a Franz diffusion cell with dermatomed human skin of 0.06 μg/(cm² h) to 0.3 μg/(cm² h), or of 0.2 μg/(cm² h) to 1.2 μg/(cm² h), or of about 0.6 μg/(cm² h) after 60 minutes.

16. Medical patch according to any one of claims 1 to 15, wherein the medical patch is a topical medical patch or a transdermal therapeutic system.

17. Medical patch according to any one of claims 1 to 16,
for use in a method of treating neuropathic pain, in particular chronic neuropathic pain, and preferably for use in a method of treating peripheral neuropathic pain, neuropathic pain associated with postherpetic neuralgia or diabetic peripheral neuropathy (DPN) of the hands and feet, post-surgical neuropathic pain, joint pain, or cancer pain.

18. Use of a medical patch according to any one of claims 1 to 16,
for the manufacture of a medicament for treating neuropathic pain, in particular chronic neuropathic pain, and preferably for the manufacture of a medicament for treating peripheral neuropathic pain, neuropathic pain associated with postherpetic neuralgia or diabetic peripheral neuropathy (DPN) of the hands or feet, post-surgical neuropathic pain, joint pain, or cancer pain.

19. A method of treating neuropathic pain, in particular chronic neuropathic pain, and preferably peripheral neuropathic pain, neuropathic pain associated with postherpetic neuralgia or diabetic peripheral neuropathy (DPN) of the hands or feet, post-surgical neuropathic pain, joint pain, or cancer pain
including applying a medical patch according to any one of claims 1 to 16 to the skin of a patient.
